(19) United States
(12) Patent Application Publication
(10) Pub. No.: US 2024/0132822 A1
(43) Pub. Date: Apr. 25, 2024

(54) Cell Culturing Device and Cell Culturing System

(71) Applicants: Terumo Kabushiki Kaisha, Tokyo (JP); Terumo BCT, Inc., Lakewood, CO (US)

(72) Inventors: Masatsugu IGARASHI, Kanagawa (JP); Thomas J. Felt, Boulder, CO (US)

(21) Appl. No.: 18/401,976

(22) Filed: Jan. 2, 2024

Related U.S. Application Data
(63) Continuation of application No. PCT/JP22/35072, filed on Sep. 21, 2022.

(30) Foreign Application Priority Data
Sep. 29, 2021 (JP) 2021-160264

(51) Int. Cl. C12M 1/12; C12M 1/34; C12M 3/00

(52) U.S. Cl. CPC C12M 25/10; C12M 23/48; C12M 37/00; C12M 41/26; C12M 41/32; C12M 41/34

(57) ABSTRACT

A cell culturing system includes a cell culturing device and a support device. The cell culturing device cultures cells by allowing a culture medium inside circulation flow paths connected to the bioreactor to flow inside the bioreactor. The cell culturing device is connected to the circulation flow paths and includes a waste liquid flow path in order to discard the liquid flowing through the circulation flow paths, and a waste liquid accommodation unit in which the liquid guided from the waste liquid flow path is accommodated. A sampling unit, in which the culture medium that is guided from the circulation flow paths to the waste liquid flow path is collected, is connected to the waste liquid flow path.

### CROSS-REFERENCE TO RELATED APPLICATIONS

The present application is a continuation application of the International Patent Application No. PCT/JP2022/035072 filed Sep. 21, 2022, which designated the U.S. and claims the benefits of priority from Japanese Patent Application No. JP2021-160264 filed Sep. 30, 2021. The entire disclosures of the above-identified applications are incorporated herein by reference.

### FIELD

The present invention relates to a cell culturing device and a cell culturing system.

### BACKGROUND

A cell culturing system may include a bioreactor and a sampling unit for collecting a culture medium (culture solution) inside the bioreactor. The sampling unit may include a sampling flow path connected to the bioreactor. A pump configured to draw the culture medium inside the bioreactor to the sampling flow path may be installed in the sampling flow path.

During use of the reactor to culture cells, it is necessary to maintain the interior of the bioreactor in an aseptic state. An aseptic filter is often installed between the bioreactor and the sampling flow path to maintain the aseptic state. However, the aseptic filter is often clogged after a period of use, for example, because of proteins and the like in the culture medium. If clogging of the aseptic filter occurs, sampling through the aseptic filter may become impossible. Further, when clogging of the aseptic filter takes place, a negative pressure may be generated in the sampling flow path between the aseptic filter and the pump, such that if the pump is turned off, a concern may arise that the liquid in the sampling flow path may flow back into the bioreactor.

### SUMMARY

In various aspects, the present disclosure provides a cell culturing device configured to culture cells by allowing a culture medium to flow through a circulation flow path that is connected to a bioreactor. The cell culturing device may include a waste liquid flow path that may be connected to the circulation flow path to permit a liquid flowing through the circulation flow path to be discarded, and a waste liquid accommodation unit in which the liquid guided from the waste liquid flow path is accommodated. The cell culturing device may also include a sampling unit that may be connected to the waste liquid flow path and that is configured to collect the culture medium that is guided from the circulation flow path to the waste liquid flow path.

In various aspects, the present disclosure provides a cell culturing system that includes a cell culturing device like that introduced above and a support device configured to receive the cell culturing device. The flow path of the cell culturing device includes one or more flexible portions, and the support device includes a plurality of clamps configured to apply a pressure to the one or more flexible portions so as to cause the flow path of the cell culturing device to close.

In at least one example embodiment, the sampling unit may be connected to the waste liquid flow path, and the liquid may always flow in a single direction from the bioreactor toward the waste liquid accommodation unit. Because of the single direction flow, the cell culturing device does not require an aseptic filter to maintain the aseptic state of the bioreactor, thereby simplifying the configuration of the cell culturing device. Further, since a negative pressure is not generated in the flow path as a result of clogging of an aseptic filter, it is possible to prevent liquid in the sampling unit from flowing back into the circulation flow path through the waste liquid flow path.

### DESCRIPTION OF DRAWINGS

FIG. 1 is a schematic circuit diagram of an example cell culturing system in accordance with at least one example embodiment of the present disclosure;

FIG. 2 is a schematic circuit diagram of an example sampling unit and a surrounding vicinity thereof in accordance with at least one example embodiment of the present disclosure;

FIG. 3 is a block diagram of an example controller in accordance with at least one example embodiment of the present disclosure;

FIG. 4 is a flowchart illustrating an example cell culturing method in accordance with at least one example embodiment of the present disclosure;

FIG. 5 is a diagram illustrating a culture preparation step of an example cell culturing method in accordance with at least one example embodiment of the present disclosure;

FIG. 6 is a flowchart illustrating a priming step of an example cell culturing method in accordance with at least one example embodiment of the present disclosure;

FIG. 7 is a diagram of a first circuit of a priming step of an example cell culturing method in accordance with at least one example embodiment of the present disclosure;

FIG. 8 is a diagram of a second circuit of a priming step of an example cell culturing method in accordance with at least one example embodiment of the present disclosure;

FIG. 9 is a flowchart illustrating a biosensor calibration step of an example cell culturing method in accordance with at least one example embodiment of the present disclosure;

FIG. 10 is a flowchart illustrating a first standard solution measurement step of an example cell culturing method in accordance with at least one example embodiment of the present disclosure;

FIG. 11 is a circuit explanatory diagram of a first standard solution measurement step of an example cell culturing method in accordance with at least one example embodiment of the present disclosure;

FIG. 12 is a flowchart illustrating a second standard solution measurement step of an example cell culturing method in accordance with at least one example embodiment of the present disclosure;

FIG. 13 is a circuit explanatory diagram of a second standard solution measurement step of an example cell culturing method in accordance with at least one example embodiment of the present disclosure;

FIG. 14 is a flowchart illustrating a sensor unit calibration step of an example cell culturing method in accordance with at least one example embodiment of the present disclosure;

FIG. 15 is a circuit explanatory diagram of a sensor unit calibration step of an example cell culturing method in accordance with at least one example embodiment of the present disclosure;

FIG. 16 is a flowchart illustrating a culturing step of an example cell culturing method in accordance with at least one example embodiment of the present disclosure;

FIG. 17 is a circuit explanatory diagram of a sampling step of an example cell culturing method in accordance with at least one example embodiment of the present disclosure;

FIG. 18 is a circuit explanatory diagram of a biosensor measurement step of an example cell culturing method in accordance with at least one example embodiment of the present disclosure;

FIG. 19 is a circuit explanatory diagram of a biosensor cleaning step of an example cell culturing method in accordance with at least one example embodiment of the present disclosure;

FIG. 20 is a circuit explanatory diagram of a stripping step of an example cell culturing method in accordance with at least one example embodiment of the present disclosure;

FIG. 21 is a circuit explanatory diagram of a collection step of an example cell culturing method in accordance with at least one example embodiment of the present disclosure; and

FIG. 22 is a circuit explanatory diagram illustrating another example of a cell culturing device in accordance with at least one example embodiment of the present disclosure.

### DETAILED DESCRIPTION

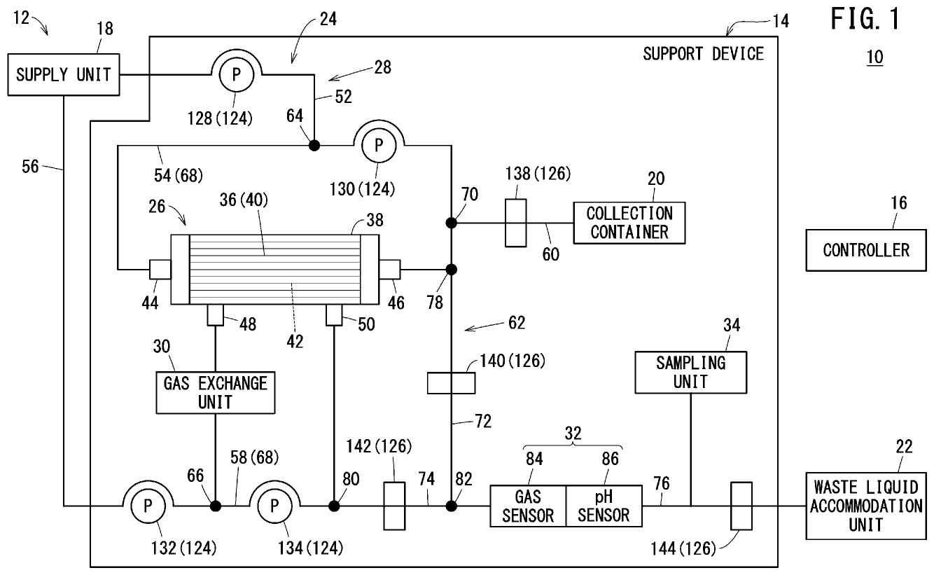

FIG. 1 illustrates an example cell culturing system 10. The cell culturing system 10 is configured to culture (propagates), within a culture medium, cells that have been separated from biological tissue. The cells used in the cell culturing system 10 may include adherent cells, planktonic cells, or a combination of adherent cells and planktonic cells. The cells used in the cell culturing system 10 may include, for example ES cells, iPS cells, mesenchymal stem cells, the like, or any combination thereof. The cells to be cultured using the cell culturing system 10 are not limited to the cell types described above.

The cell culturing system 10 includes a cell culturing device 12, a support device 14, and a controller 16. A liquid including, for example, a cell solution, a culture medium, a cleaning solution, a stripping solution, or any combination thereof may flow through the cell culturing device 12.

The cell solution may be a solution that includes cells. The culture medium may cause the cells in contact therewith to propagate. The culture medium may be selected depending on the cells to be cultured. The culture medium may include, for example, an MEM (Minimum Essential Media). The cleaning solution may be selected to clean the interior of the cell culturing device 12. The cleaning solution may include, for example, water, a buffer solution, a physiological saline solution, the like, or any combination thereof. the buffer solution may include, for example, PBS (Phosphate Buffered Salts), TBS (Tris-Buffered Saline), the like, or any combination thereof. The stripping solution may be selected to strip the cells from a later-described bioreactor 26 of the cell culturing device 12. The stripping solution may include, for example, trypsin, an EDTA solution, or any combination thereof. The culture medium, the cleaning solution, and the stripping solution are not limited to the liquids described above.

The cell culturing device 12 may be discarded after being used one time (e.g., every time that a predetermined number of cells have been cultured). That is, the cell culturing device 12 may be a disposable product. The cell culturing device 12 may include, for example, a supply unit 18, a collection container 20, a waste liquid accommodation unit 22, and a culturing body 24.

The supply unit 18 may be configured to supply the cell solution, the culture medium, the cleaning solution, and/or the stripping solution to the culturing body 24. The collection container 20 may be configured to collect the cells that are cultured in the culturing body 24. The waste liquid accommodation unit 22 may be configured to accommodate the waste liquid that is generated in the culturing body 24. The collection container 20 and/or the waste liquid accommodation unit 22 may include, for example, a medical bag, which may be obtained by molding a soft resin material into a bag-like shape. The soft resin material may include, for example, cited polyvinyl chloride, polyolefin, or a combination of polyvinyl chloride and polyolefin. In other example embodiments, the collection container 20 and/or the waste liquid accommodation unit 22 may include a tank or the like formed using, for example, a hard resin.

The culturing body 24 may include a bioreactor 26, a culturing circuit 28, a gas exchange unit 30, a sensor unit 32, and a sampling unit 34.

The bioreactor 26 may include a plurality of hollow fiber membranes 36 and a cylindrical housing 38. The plurality of hollow fiber membranes 36 may be disposed in the housing 38. One end (e.g., a first end) of the respective hollow fiber membranes 36 may be fixed to one end (e.g., a first end) of the housing 38. Another end (e.g., a second end) of the respective hollow fiber membranes 36 may be fixed to another end (e.g., second end) of the housing 38.

The respective hollow fiber membranes 36 may include a polymer material. The polymer material may include, for example, polypropylene, polyolefin resin, polysulfone, polyether sulfone, polyacrylonitrile, polytetrafluoroethylene, polystyrene, polymethylmethacrylate, cellulose acetate, cellulose triacetate, regenerated cellulose, the like, or any combination thereof. However, the material constituting the respective hollow fiber membranes 36 is not limited to the aforementioned materials.

The bioreactor 26 may include a first region 40 and a second region 42. The first region 40 may be defined by inner holes of a plurality of hollow fiber membranes 36. The second region 42 may be defined by a space between an inner peripheral surface of the housing 38 and outer peripheral surfaces of the plurality of hollow fiber membranes 36. Although not illustrated, it should be appreciated that each of the hollow fiber membranes 36 may include a plurality pores therein. The first region 40 and the second region 42 may communicate with each other through the plurality of pores of the respective hollow fiber membranes 36. The diameter of the pores may be of a size that allows small molecules (for example, water, ions, oxygen, lactic acid, etc.) to pass therethrough, while preventing the passage of macromolecules (for example, cells, etc.) therethrough. In at least one example embodiment, the diameter of the pores may be greater than or equal to 0.005 micrometers (μm) and less than or equal to 10 micrometers (μm).

The housing 38 may include a first inlet port 44, a first outlet port 46, a second inlet port 48, and a second outlet port 50. The first inlet port 44 may be installed at one end of the housing 38. The first inlet port 44 may communicate with the first region 40 via an inlet positioned at one end of the plurality of hollow fiber membranes 36. The first outlet port 46 may be installed at another end of the housing 38. The first outlet port 46 may communicate with the first region 40 via an outlet positioned at the other end of the plurality of hollow fiber membranes 36.

The second inlet port 48 and the second outlet port 50 may be installed on an outer peripheral surface of the housing 38. The second inlet port 48 may be positioned between a center of the housing 38 and the first inlet port 44 in the longitudinal direction of the housing 38. The second outlet port 50 may be positioned between the center of the housing 38 and the first outlet port 46 in the longitudinal direction of the housing 38. Each of the second inlet port 48 and the second outlet port 50 may communicate with the second region 42.

The culturing circuit 28 may include flow paths which are extended in a linear shape. For example, in at least one example embodiment, the culturing circuit 28 may include a plurality of tubes through which the liquids flow. The respective tubes may be formed of a soft resin material. A wall portion of the flow paths (culturing circuit 28) of the cell culturing device 12 may be flexible.

The culturing circuit 28, however, is not limited to the configuration described above. In at least one example embodiment, the culturing circuit 28 may include, for example, a sheet member including the flow paths therein through which the liquids flow. The sheet member may be formed of two sheets made of a soft resin material which are stacked on each other in a thickness direction. Locations within the two sheets other than portions thereof that make up the flow paths may be joined (e.g., fusion bonded) mutually to each other. Within the two sheets, flow path wall parts that make up the flow paths are not joined to each other. Within the sheet members, the flow path wall parts preferably bulge outward in a natural state in which liquid is not flowing through the flow paths. Extra portions on both sides of the sheet member in directions intersecting the flow paths may be cut off.

The culturing circuit 28 may include a first supply flow path 52, a first circulation flow path 54, a second supply flow path 56, a second circulation flow path 58, a collection flow path 60, and a waste liquid flow path 62. One end of the first supply flow path 52 may be connected to the supply unit 18. The supply unit 18 may be configured to supply the cell solution, the culture medium, the cleaning solution, and/or the stripping solution, for example, one at a time at a predetermined timing, to the first supply flow path 52. Another end of the first supply flow path 52 may merge with the first circulation flow path 54.

Within the first circulation flow path 54, a first merging section 64, which is a portion to which the first supply flow path 52 may be connected, may be positioned at an intermediate portion in a direction in which the first circulation flow path 54 extends. One end (e.g., a first end) of the first circulation flow path 54 may be connected to the first inlet port 44. Another end (e.g., a second end) of the first circulation flow path 54 may be connected to the first outlet port 46. The first circulation flow path 54 may communicate with the inner holes (the first region 40) of the plurality of hollow fiber membranes 36.

One end (e.g., a first end) of the second supply flow path 56 may be connected to the supply unit 18. The supply unit 18 may supply the culture medium and the cleaning solution, for example one at a time at a predetermined timing, to the second supply flow path 56. Another end (e.g., a second end) of the second supply flow path 56 may merge with the second circulation flow path 58.

Within the second circulation flow path 58, a second merging section 66, which is a portion to which the second supply flow path 56 may be connected, may be positioned at an intermediate portion in a direction in which the second circulation flow path 58 extends. One end (e.g., a first end) of the second circulation flow path 58 may be connected to the second inlet port 48. Another end (e.g., a second end) of the second circulation flow path 58 may be connected to the second outlet port 50. The second circulation flow path 58 may communicate with the space (the second region 42) between the plurality of hollow fiber membranes 36 and the housing 38. Hereinafter, the first circulation flow path 54 and the second circulation flow path 58 may be collectively referred to as “circulation flow paths 68”.

The collection flow path 60 may extend from the first circulation flow path 54. Within the first circulation flow path 54, a collection branching section 70, which is a portion to which the collection flow path 60 may be connected, may be positioned between the first merging section 64 and the first outlet port 46 in the first circulation flow path 54. An extending end of the collection flow path 60 may be connected to the collection container 20.

The waste liquid flow path 62 may be a flow path for discarding the liquid that flows through the circulation flow paths 68. The waste liquid flow path 62 may include a first waste liquid flow path 72, a second waste liquid flow path 74, and a third waste liquid flow path 76. The first waste liquid flow path 72 may extend from the first circulation flow path 54. Within the first circulation flow path 54, a first branching section 78, which is a portion to which the first waste liquid flow path 72 may be connected, may be positioned between the first outlet port 46 and the collection branching section 70 in the first circulation flow path 54.

The second waste liquid flow path 74 may extend from the second circulation flow path 58. Within the second circulation flow path 58, a second branching section 80, which is a portion to which the second waste liquid flow path 74 may be connected, may be positioned between the second merging section 66 and the second outlet port 50 in the second circulation flow path 58.

An extending end of the first waste liquid flow path 72 and an extending end of the second waste liquid flow path 74 may be connected to one end of the third waste liquid flow path 76. That is, the one end (e.g., a first end) of the third waste liquid flow path 76 may be an intermediate merging section 82 where the extending end of the first waste liquid flow path 72 and the extending end of the second waste liquid flow path 74 merge. Another end (e.g., a second end) of the third waste liquid flow path 76 may be connected to the waste liquid accommodation unit 22.

The gas exchange unit 30 may be installed in the second circulation flow path 58 between the second merging section 66 and the second inlet port 48. The gas exchange unit 30 may allow a gas having predetermined components to pass through the liquid (culture medium) that flows through the second circulation flow path 58. The gas used in the gas exchange unit 30 may include, for example, components that are similar to those of natural air. That is, the gas may include nitrogen, oxygen, carbon dioxide, or any combination thereof. In at least one example embodiment, the gas may include, for example, 75% nitrogen, 20% oxygen, and 5% carbon dioxide by volume.

The sensor unit 32 may be installed in the third waste liquid flow path 76. The sensor unit 32 may be an integrally molded product. The sensor unit 32 may include a gas sensor 84 and/or a pH sensor 86. The gas sensor 84 may be configured to measure a gas concentration of the liquid flowing through the third waste liquid flow path 76. In at least one example embodiment, the gas sensor 84 includes, for example, an oxygen sensor and/or a carbon dioxide sensor. The oxygen sensor may be configured to measure an oxygen concentration of the liquid flowing through the third waste liquid flow path 76. The carbon dioxide sensor may be configured to measure a carbon dioxide concentration of the liquid flowing through the third waste liquid flow path 76. The pH sensor 86 may be configured to measure a pH (e.g., hydrogen ion index) of the liquid flowing through the third waste liquid flow path 76. The gas sensor 84 and the pH sensor 86 may be non-enzyme sensors on which a sterilization treatment can be performed. Such a sensor unit 32 can be subjected to the sterilization treatment, for example, in a state of being installed in the middle of a tube both ends of which are sealed. In such instances, as for the sensor unit 32 which has been subjected to such a sterilization treatment, both ends of the tube can be connected by an aseptic joining device to an appropriate portion of the culturing circuit 28.

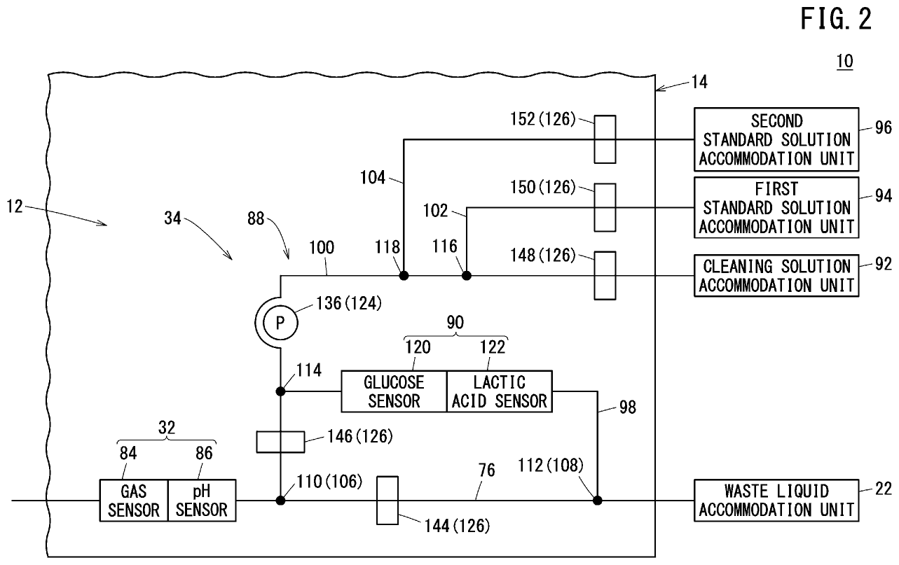

The sampling unit 34 may be connected to a portion within the third waste liquid flow path 76 between the sensor unit 32 and the waste liquid accommodation unit 22. As illustrated in FIG. 2, the sampling unit 34 may include a measurement circuit 88, a biosensor 90, a cleaning solution accommodation unit 92, a first standard solution accommodation unit 94, and a second standard solution accommodation unit 96.

The measurement circuit 88 may include flow paths that extended in a linear shape. The measurement circuit 88 may include a plurality of tubes through which the liquids flow. The respective tubes may be formed of a soft resin material. However, the measurement circuit 88 may include, for example, a sheet member including the flow paths therein through which the liquids flow. The sheet member may be configured in the same manner as the sheet member constituting the aforementioned culturing circuit 28. The measurement circuit 88 may include a sampling flow path 98, a first introduction flow path 100, a second introduction flow path 102, and a third introduction flow path 104.

The sampling flow path 98 may have a first end 106 and a second end 108. The first end 106 may be one end of the sampling flow path 98. The second end 108 may be another end of the sampling flow path 98. Each of the first end 106 and the second end 108 may be connected to the third waste liquid flow path 76. Within the third waste liquid flow path 76, a third branching section 110, which is a portion to which the first end 106 of the sampling flow path 98 may be connected, may be positioned between the sensor unit 32 and the waste liquid accommodation unit 22 in the third waste liquid flow path 76. Within the third waste liquid flow path 76, a third merging section 112, which is a portion to which the second end 108 of the sampling flow path 98 may be connected, may be positioned between the third branching section 110 and the waste liquid accommodation unit 22 in the third waste liquid flow path 76. The sampling flow path 98 may be aseptically joined to the third waste liquid flow path 76 at the positions of the third branching section 110 and the third merging section 112. Although not illustrated, it should be appreciated that, in at least one example embodiment, the sampling flow path 98 may be connected to the third waste liquid flow path 76 via connectors, for example, at the positions of the third branching section 110 and the third merging section 112.

One end (e.g., a first end) of the first introduction flow path 100 may be connected to the cleaning solution accommodation unit 92. Another end (e.g., a second end) of the first introduction flow path 100 may be connected to the sampling flow path 98. In the sampling flow path 98, a fourth merging section 114, which is a portion to which the first introduction flow path 100 may be connected, may be positioned at an intermediate portion in a direction in which the sampling flow path 98 extends.

One end (e.g., a first end) of the second introduction flow path 102 may be connected to the first standard solution accommodation unit 94. Another end (e.g., a second end) of the second introduction flow path 102 may be connected to the first introduction flow path 100. Within the first introduction flow path 100, a fifth merging section 116, which is a portion to which the second introduction flow path 102 may be connected, may be positioned at an intermediate portion in a direction in which the first introduction flow path 100 extends.

One end (e.g., a first end) of the third introduction flow path 104 may be connected to the second standard solution accommodation unit 96. Another end (e.g., a second end) of the third introduction flow path 104 may be connected to the first introduction flow path 100. Within the first introduction flow path 100, a sixth merging section 118, which is a portion to which the third introduction flow path 104 may be connected, may be positioned between the fourth merging section 114 and the fifth merging section 116 in the first introduction flow path 100.

The biosensor 90 may be installed in the sampling flow path 98 in a portion thereof between the fourth merging section 114 and the second end 108. The biosensor 90 may be an integrally molded enzyme sensor. The biosensor 90 may include, for example, a glucose sensor 120 and/or a lactic acid sensor 122. The glucose sensor 120 and/or the lactic acid sensor 122 may be placed in contact with the liquid flowing through the sampling flow path 98. The glucose sensor 120 may be configured to measure a glucose concentration of the liquid flowing through the sampling flow path 98. The lactic acid sensor 122 may be configured to measure a lactic acid concentration of the liquid flowing through the sampling flow path 98.

The biosensor 90 may include a glutamic acid sensor that is configured to measure a glutamic acid concentration of the liquid flowing through the sampling flow path 98. The biosensor 90 is not limited to being an enzyme sensor and, in at least one example embodiment, may include a non-enzyme sensor.

The cleaning solution accommodation unit 92, the first standard solution accommodation unit 94, and the second standard solution accommodation unit 96 may include medical bags, for example, in the same manner as the waste liquid accommodation unit 22, as described above. In at least one example embodiment, the cleaning solution accommodation unit 92, the first standard solution accommodation unit 94, and/or the second standard solution accommodation unit 96 may include tanks or the like formed using a hard resin.

The cleaning solution may be accommodated in the cleaning solution accommodation unit 92. The cleaning solution may include a solution for cleaning the biosensor 90. In at least one example embodiment, the cleaning solution may include, for example, a solution similar to the cleaning solution supplied from the aforementioned supply unit 18 to the culturing circuit 28.

A first standard solution may be accommodated in the first standard solution accommodation unit 94. The first standard solution may be a solution used for calibrating the biosensor 90. A glucose concentration of the first standard solution may be set to a first standard glucose concentration. A lactic acid concentration of the first standard solution may be set to a first standard lactic acid concentration.

A second standard solution may be accommodated in the second standard solution accommodation unit 96. The second standard solution may be a solution used for calibrating the biosensor 90. A glucose concentration of the second standard solution may be set to a second standard glucose concentration. The second standard glucose concentration may be a value that differs from the first standard glucose concentration. A lactic acid concentration of the second standard solution may be set to a second lactic acid concentration. The second standard lactic acid concentration may be a value that differs from the first standard lactic acid concentration.

As illustrated in FIGS. 1 and 2, the cell culturing device 12 may be set on the support device 14. The support device 14 may include a cassette that supports the cell culturing device 12. The support device 14 may be a reusable product that is capable of being used a plurality of times.

The support device 14 may be equipped with a plurality of pumps 124 and a plurality of clamps 126. Each of the plurality of pumps 124 may impart a flowing force to the liquids inside the flow paths by squeezing the flow path wall parts of the cell culturing device 12. Although not illustrated, it should be appreciated that in at least one example embodiment, each of the plurality of pumps 124 may include a pressing member. The pressing member may include, for example, a rotating member and a plurality of pressing rollers. The plurality of pressing rollers may be attached to an outer circumferential portion of the rotating member. The plurality of pressing rollers may be arranged at intervals with spaces left therebetween in the circumferential direction of the rotating member. Each of the pressing rollers may rub against the outer surfaces of the flow path wall parts of the cell culturing device 12.

The plurality of pumps 124 may include, for example, a first supply pump 128, a first circulation pump 130, a second supply pump 132, a second circulation pump 134, and/or an introduction pump 136.

As illustrated in FIG. 1, in a state in which the cell culturing device 12 is set on the support device 14 (hereinafter, simply referred to as a “set state”), a flow path wall part of an intermediate portion in a direction in which the first supply flow path 52 extends may be mounted in the first supply pump 128. The first supply pump 128 may be configured to apply a flowing force to the liquid inside the first supply flow path 52 in a direction from the supply unit 18 toward the first circulation flow path 54.

In the set state, a flow path wall part of a portion between the first merging section 64 and the collection branching section 70 in the first circulation flow path 54 may be mounted in the first circulation pump 130. The first circulation pump 130 may be configured to apply a flowing force to the liquid inside the first circulation flow path 54 in a direction toward the first inlet port 44. Moreover, the first circulation pump 130 may also be configured to apply a flowing force to the liquid inside the first circulation flow path 54 in a direction toward the first outlet port 46.

In the set state, a flow path wall part of an intermediate portion in a direction in which the second supply flow path 56 extends may be mounted in the second supply pump 132. The second supply pump 132 may be configured to apply a flowing force to the liquid inside the second supply flow path 56 in a direction from the supply unit 18 toward the second circulation flow path 58.

In the set state, a flow path wall part of a portion between the second merging section 66 and the second branching section 80 in the second circulation flow path 58 may be mounted in the second circulation pump 134. The second circulation pump 134 may be configured to apply a flowing force to the liquid inside the second circulation flow path 58 in a direction toward the second inlet port 48. Moreover, the second circulation pump 134 may also apply a flowing force to the liquid inside the second circulation flow path 58 in a direction toward the second outlet port 50.

As illustrated in FIG. 2, in the set state, a flow path wall part of an intermediate portion in a direction in which the first introduction flow path 100 extends may be mounted in the introduction pump 136. The introduction pump 136 may apply a flowing force to the liquid inside the first introduction flow path 100 in a direction toward the sampling flow path 98.

As illustrated in FIGS. 1 and 2, the plurality of clamps 126 may be on-off valves that serve to close the internal flow paths by pressing on the outer surfaces of the flow path wall parts of the cell culturing device 12 toward the inner surfaces. The plurality of clamps 126 may include, for example, a collection clamp 138, a first waste liquid clamp 140, a second waste liquid clamp 142, a third waste liquid clamp 144, a sampling clamp 146, a first introduction clamp 148, a second introduction clamp 150, and/or a third introduction clamp 152.

As illustrated in FIG. 1, in the set state, a flow path wall part of an intermediate portion in a direction in which the collection flow path 60 extends may be mounted in the collection clamp 138. The collection clamp 138 may be configured to open and close the intermediate portion in the direction in which the collection flow path 60 extends. In the set state, a flow path wall part of an intermediate portion in a direction in which the first waste liquid flow path 72 extends may be mounted in the first waste liquid clamp 140. The first waste liquid clamp 140 may be configured to open and close the intermediate portion in the direction in which the first waste liquid flow path 72 extends. A flow path wall part of an intermediate portion in a direction in which the second waste liquid flow path 74 extends may be mounted in the second waste liquid clamp 142. The second waste liquid clamp 142 may be configured to open and close the intermediate portion in the direction in which the second waste liquid flow path 74 extends.

As illustrated in FIG. 2, in the set state, a flow path wall part of a portion between the first end 106 and the fourth merging section 114 in the sampling flow path 98 may be mounted in the sampling clamp 146. The sampling clamp 146 may be configured to open and close the portion of the sampling flow path 98 between the first end 106 and the fourth merging section 114.

In the set state, a flow path wall part of a portion between the cleaning solution accommodation unit 92 and the fifth merging section 116 in the first introduction flow path 100 may be mounted in the first introduction clamp 148. The first introduction clamp 148 may be configured to open and close the portion of the first introduction flow path 100 between the cleaning solution accommodation unit 92 and the fifth merging section 116.

In the set state, a flow path wall part of an intermediate portion in a direction in which the second introduction flow path 102 extends may be mounted in the second introduction clamp 150. The second introduction clamp 150 may be configured to open and close the intermediate portion in the direction in which the second introduction flow path 102 extends. In the set state, a flow path wall part of an intermediate portion in a direction in which the third introduction flow path 104 extends may be mounted in the third introduction clamp 152. The third introduction clamp 152 may be configured to open and close the intermediate portion in the direction in which the third introduction flow path 104 extends.

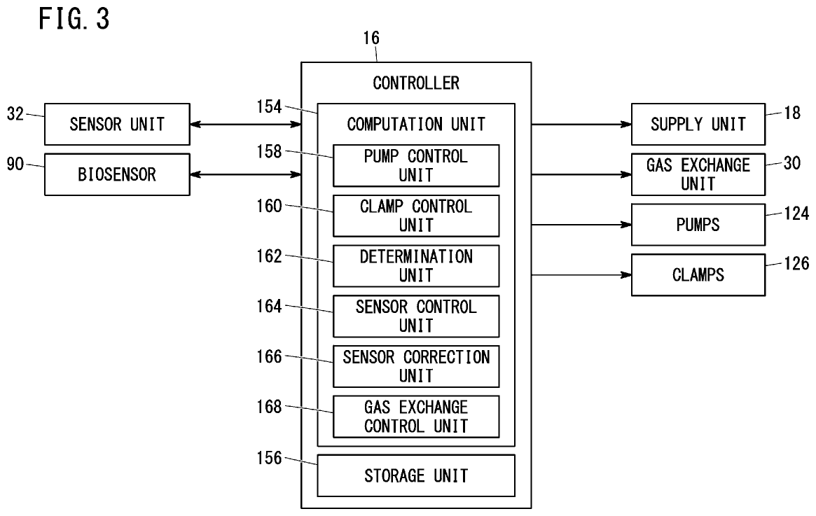

As illustrated in FIG. 3, the supply unit 18, the gas exchange unit 30, the sensor unit 32, the biosensor 90, the plurality of pumps 124, and/or the plurality of clamps 126 may be connected wirelessly or over wires to the controller 16. The controller 16 may include, for example, a computation unit 154 (processing unit) and a storage unit 156. The computation unit 154 may include, for example, a processor (processing circuit), such as a CPU (Central Processing Unit), a GPU (Graphics Processing Unit), or the like.

The computation unit 154 may include a pump control unit 158, a clamp control unit 160, a determination unit 162, a sensor control unit 164, a sensor correction unit 166, and/or a gas exchange control unit 168. By executing a program stored in the storage unit 156, the computation unit 154 may be configured to realize the pump control unit 158, the clamp control unit 160, the determination unit 162, the sensor control unit 164, the sensor correction unit 166, and/or the gas exchange control unit 168.

The computation unit 154 may realize at least a portion of the pump control unit 158, the clamp control unit 160, the determination unit 162, the sensor control unit 164, the sensor correction unit 166, and/or the gas exchange control unit 168 by way of an integrated circuit. The integrated circuit may include, for example, an ASIC (Application Specific Integrated Circuit), an FPGA (Field-Programmable Gate Array), the like, or any combination thereof.

The storage unit 156 may include a volatile memory and a non-volatile memory. The volatile memory may include, for example, a RAM (Random Access Memory) or the like. The volatile memory may be used as a working memory of the processor and data and the like required for carrying out processing or calculations may be temporarily stored therein. The non-volatile memory may include, for example, a ROM (Read Only Memory), a flash memory, the like, or any combination thereof. The non-volatile memory may be used as a storage memory. Programs, tables, maps, etc. may be stored in the non-volatile memory. At least a portion of the storage unit 156 may be incorporated in the processor or the integrated circuit, such as were described above.

The pump control unit 158 may be configured to control the plurality of pumps 124. The clamp control unit 160 may be configured to control the plurality of clamps 126. The determination unit 162 may be configured to carry out a predetermined determination process. The sensor control unit 164 may be configured to control the biosensor 90 and the sensor unit 32. The sensor correction unit 166 may be configured to perform corrections in order to calibrate the biosensor 90 and the sensor unit 32. The gas exchange control unit 168 may be configured to control the gas exchange unit 30.

In various aspects, the present disclosure provides cell culturing methods where cell culturing systems, like the cell culturing system 10, may be used.

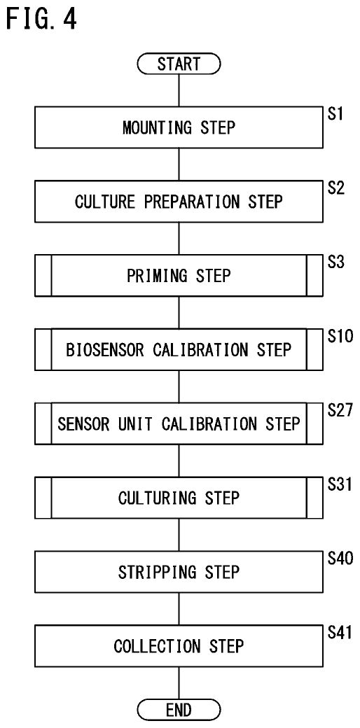

As illustrated in FIG. 4, the cell culturing method may include, for example, a mounting step, a culture preparation step, a priming step, a biosensor calibration step, a sensor unit calibration step, a culturing step, a stripping step, and a collection step.

In the mounting step (step S1), the cell culturing device 12 may be set on the support device 14. Thereafter, the clamp control unit 160 may control the plurality of clamps 126. For example, the clamp control unit 160 may cause one or more of the plurality of clamps 126 to be in a closed state, thereby causing portions within the flow paths of the cell culturing device 12 corresponding to the plurality of clamps 126 to be closed.

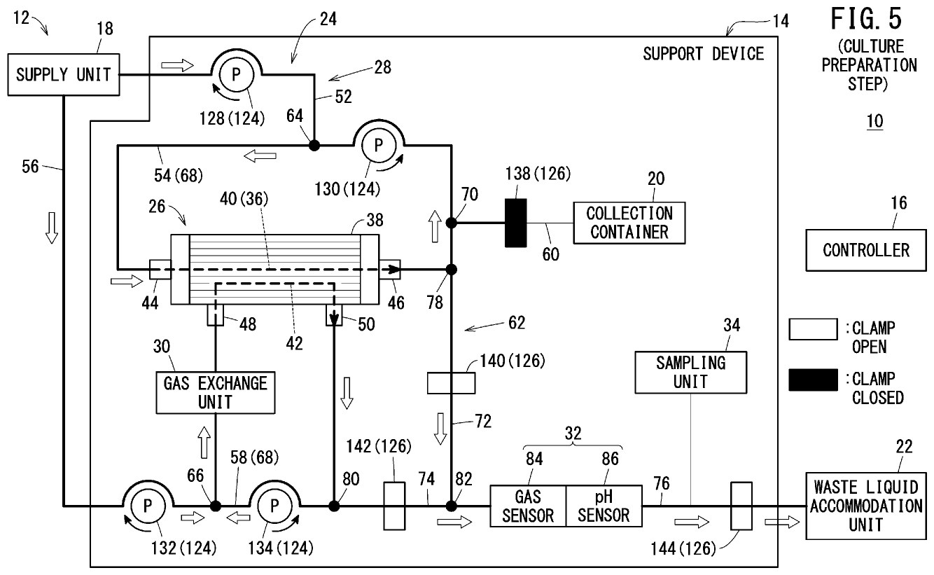

After the cell culturing device 12 is set on the support device 14, the method may proceed to the culture preparation step (step S2). As illustrated in FIG. 5, during the culture preparation step, the clamp control unit 160 may control the first waste liquid clamp 140, the second waste liquid clamp 142, and the third waste liquid clamp 144. The clamp control unit 160 may place the first waste liquid clamp 140, the second waste liquid clamp 142, and the third waste liquid clamp 144 in an open state, such that the first waste liquid flow path 72, the second waste liquid flow path 74, and the third waste liquid flow path 76 are opened. The controller 16 may control the supply unit 18 so to cause the cleaning solution to be supplied from the supply unit 18 to the first supply flow path 52 and the second supply flow path 56.

The pump control unit 158 may turn on the first supply pump 128 and the first circulation pump 130. Upon doing so, the cleaning solution may be introduced from the supply unit 18 into the first merging section 64 of the first circulation flow path 54 via the first supply flow path 52. The cleaning solution having been introduced into the first merging section 64 may flow from the first inlet port 44 through the first region 40 and may be guided to the first outlet port 46. The cleaning solution that is guided to the first outlet port 46 may be returned to the first merging section 64 via the first branching section 78 and the collection branching section 70 of the first circulation flow path 54. The cleaning solution may circulate in an annular flow path that includes the first circulation flow path 54, the first inlet port 44, the first region 40, and the first outlet port 46. At this time, a portion of the cleaning solution flowing through the first circulation flow path 54 may flow from the first branching section 78 into the first waste liquid flow path 72.

The pump control unit 158 may turn on the second supply pump 132 and the second circulation pump 134. Upon doing so, the cleaning solution may be introduced from the supply unit 18 into the second merging section 66 of the second circulation flow path 58 via the second supply flow path 56. The cleaning solution having been introduced into the second merging section 66 may flow from the second inlet port 48 through the second region 42 and may be guided to the second outlet port 50. The cleaning solution that is guided to the second outlet port 50 may be returned to the second merging section 66 via the second branching section 80 of the second circulation flow path 58. The cleaning solution may circulate in an annular flow path including the second circulation flow path 58, the second inlet port 48, the second region 42, and the second outlet port 50. At this time, a portion of the cleaning solution flowing through the second circulation flow path 58 may flow from the second branching section 80 into the second waste liquid flow path 74.

The cleaning solution flowing through the first waste liquid flow path 72 and the cleaning solution flowing through the second waste liquid flow path 74 may merge together at the intermediate merging section 82. The cleaning solution having been merged at the intermediate merging section 82 may be guided to the waste liquid accommodation unit 22 via the third waste liquid flow path 76. In accordance therewith, the culturing circuit 28 and the bioreactor 26 may be filled with the cleaning solution.

The controller 16 may control the supply unit 18 to cause the culture medium to be supplied from the supply unit 18 to the first supply flow path 52 and the second supply flow path 56. In accordance therewith, the culturing circuit 28 and the bioreactor 26 may be filled with the culture medium. That is, the cleaning solution existing in the culturing circuit 28 and the bioreactor 26 may be replaced by the culture medium.

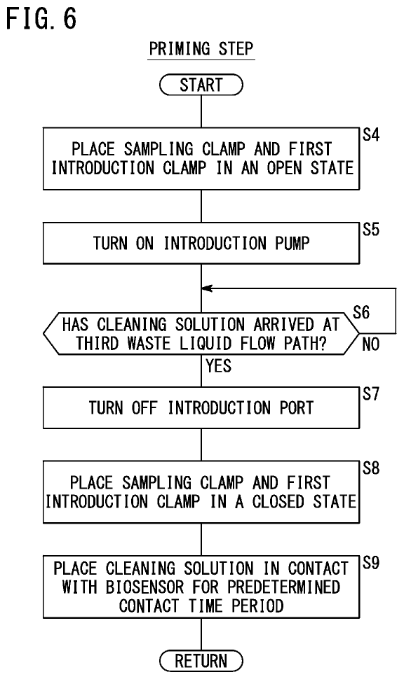

The priming step (step S3 of FIG. 4) may then be carried out. As illustrated in FIG. 6, during the priming step the clamp control unit 160 may control the sampling clamp 146 and the first introduction clamp 148 to place them in an open state, and in the sampling flow path 98, a portion between the third branching section 110 and the fourth merging section 114 may be opened together with opening the first introduction flow path 100 (step S4). Further, the pump control unit 158 may turn on the introduction pump 136 (step S5).

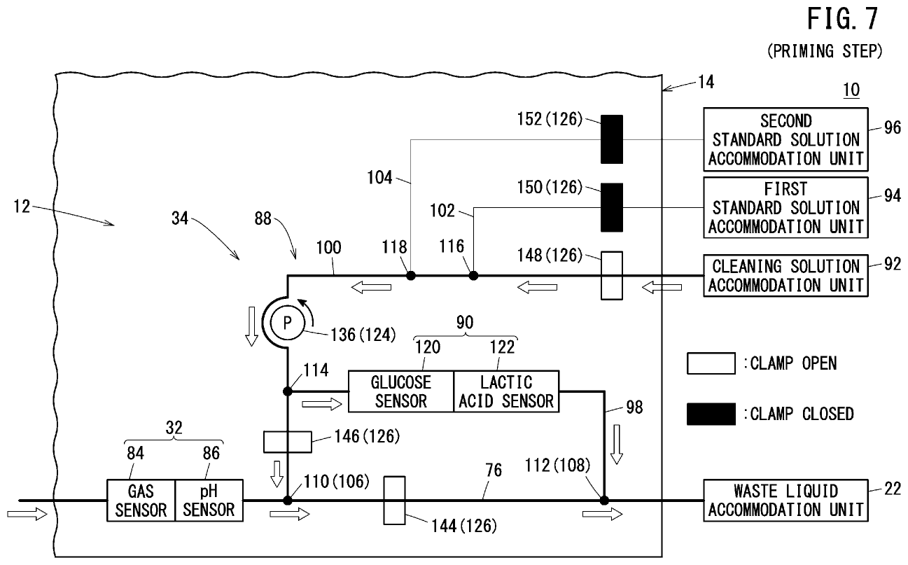

Upon doing so, as illustrated in FIG. 7, the cleaning solution may flow from the cleaning solution accommodation unit 92 to the waste liquid accommodation unit 22 via the first introduction flow path 100, the sampling flow path 98, and the third waste liquid flow path 76. At this time, the determination unit 162 may determine whether or not the cleaning solution has arrived at the third waste liquid flow path 76 (step S6 in FIG. 6).

In at least one example embodiment, the determination unit 162 may determine, for example, whether or not an elapsed time period since the introduction pump 136 has been turned on has reached a predetermined cleaning solution introduction time period. The cleaning solution introduction time period may be set based on the shape of the sampling unit 34, the size of the sampling unit 34, the flow rate of the cleaning solution, and/or the like. The cleaning solution introduction time period may be stored in the storage unit 156. Moreover, the determination unit 162 may determine whether or not the cleaning solution has arrived at the third waste liquid flow path 76 based on a detection signal of a cleaning solution sensor that is configured to detect the cleaning solution. The cleaning solution sensor may be arranged, for example, in a portion between the third merging section 112 and the waste liquid accommodation unit 22 in the third waste liquid flow path 76.

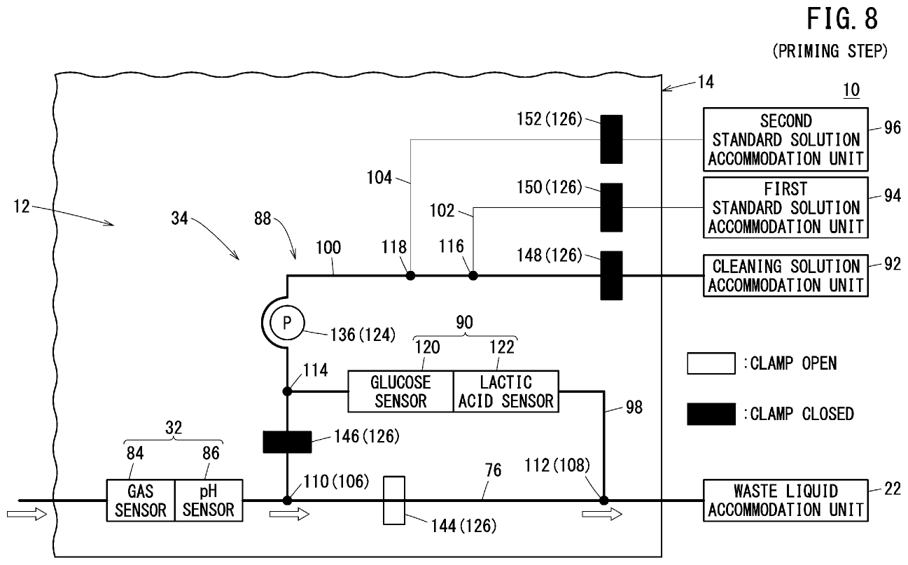

As illustrated in FIG. 6, when it is determined, for example, by the determination unit 162, that the cleaning solution has not arrived at the third waste liquid flow path 76 (step S6: NO), step S6 may be performed again. When it is determined, for example, by the determination unit 162, that the cleaning solution has arrived at the third waste liquid flow path 76 (step S6: YES), the pump control unit 158 may turn off the introduction pump 136 (step S7). Further, the clamp control unit 160 may place the sampling clamp 146 and the first introduction clamp 148 in a closed state, and in the sampling flow path 98, the portion between the third branching section 110 and the fourth merging section 114 may be closed together with closing the first introduction flow path 100 (step S8). Accordingly, flowing of the cleaning solution in the biosensor 90 is stopped (see FIG. 8).

The cleaning solution may be placed in contact with the biosensor 90 for a predetermined contact time period (step S9). In at least one example embodiment, the contact time period may be set, for example, to be greater than or equal to one hour. The cleaning solution may permeate into the entire portion within the biosensor 90 that comes into contact with the cleaning solution. The contact time period can be set in any appropriate manner. The contact time period may be stored in the storage unit 156. In accordance therewith, the priming step may be brought to an end.

When the priming step is carried out, the culture medium may continuously flow through the waste liquid flow path 62 toward the waste liquid accommodation unit 22. Therefore, during the priming step, it may be possible to effectively prevent the cleaning solution from flowing back into the circulation flow paths 68.

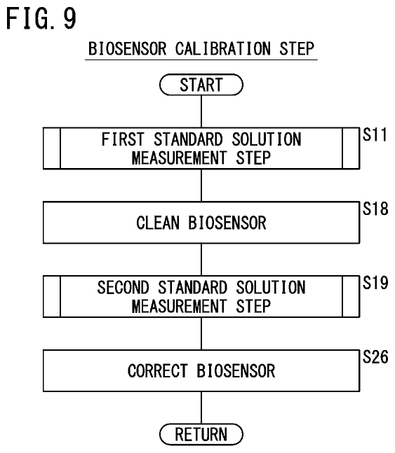

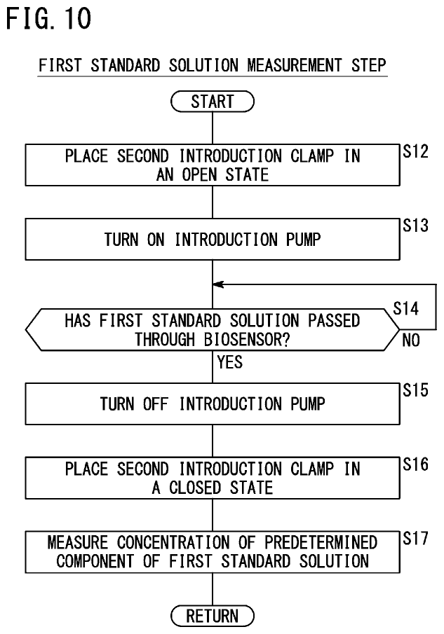

After the priming step, the biosensor calibration step (step S10 of FIG. 4) may be carried out. More specifically, in the biosensor calibration step, as illustrated in FIG. 9, the first standard solution measurement step (step S11) may be carried out. As illustrated in FIG. 10, in the first standard solution measurement step, the clamp control unit 160 may control the second introduction clamp 150 to be placed in an open state, thereby causing the second introduction flow path 102 to open (step S12). Further, the pump control unit 158 may turn on the introduction pump 136 (step S13).

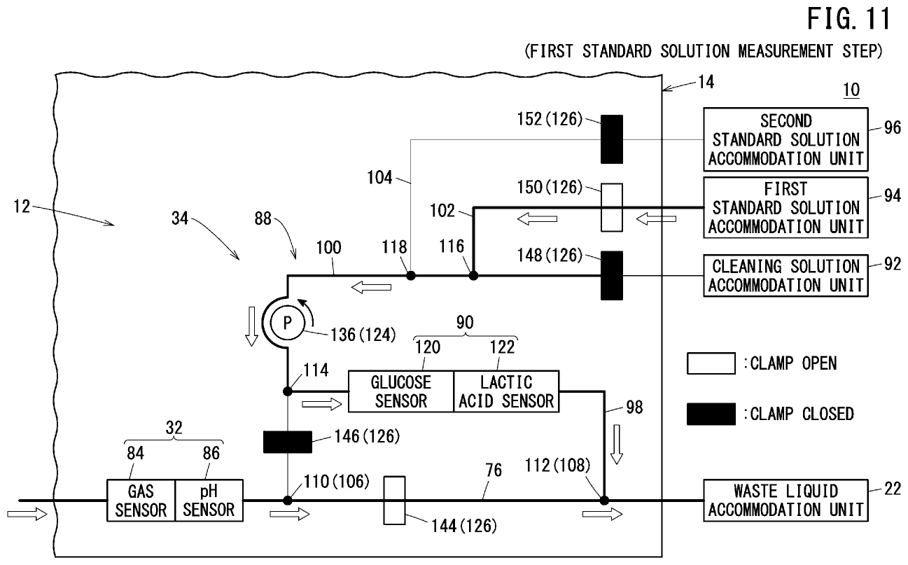

Upon doing so, as illustrated in FIG. 11, the first standard solution may flow from the first standard solution accommodation unit 94 into the waste liquid accommodation unit 22 via the second introduction flow path 102, the first introduction flow path 100, the sampling flow path 98, and the third waste liquid flow path 76. At this time, the determination unit 162 may determine whether or not the first standard solution has passed through the biosensor 90 (step S14).

In at least one example embodiment, the determination unit 162 may determine, for example, whether or not an elapsed time period since the introduction pump 136 has been turned on has reached a first standard solution introduction time period. The first standard solution introduction time period may be set based on the shape of the sampling unit 34, the size of the sampling unit 34, the flow rate of the first standard solution, and/or the like. The first standard solution introduction time period may be stored in the storage unit 156. Moreover, the determination unit 162 may determine whether or not the first standard solution has passed through the biosensor 90 based on a detection signal of a first standard solution sensor that is configured to detect the first standard solution. The first standard solution sensor may be arranged, for example, in the sampling flow path 98 in a portion thereof between the biosensor 90 and the third merging section 112. However, in other example embodiments, the first standard solution sensor may be arranged in the third waste liquid flow path 76 in a portion thereof between the third merging section 112 and the waste liquid accommodation unit 22.

As illustrated in FIG. 10, when it is determined, for example, by the determination unit 162, that the first standard solution has not passed through the biosensor 90 (step S14: NO), the process of step S14 may be performed again. When it is determined by the determination unit 162 that the first standard solution has passed through the biosensor 90 (step S14: YES), the pump control unit 158 may turn off the introduction pump 136 (step S15). Further, the clamp control unit 160 may cause the second introduction clamp 150 to be placed in a closed state, thereby causing the second introduction flow path 102 to close (step S16) and stopping the flow of the first standard solution of the biosensor 90.

The sensor control unit 164 may control the biosensor 90 to measure the concentration of a predetermined component of the first standard solution (step S17). In at least one example embodiment, the sensor control unit 164 may control the glucose sensor 120 to measure the glucose concentration of the first standard solution. The glucose sensor 120 may transmit a first measured glucose concentration, which is the measured glucose concentration of the first standard solution, or an indicator thereof, to the controller 16.

The sensor control unit 164 may control the lactic acid sensor 122 to measure the lactic acid concentration of the first standard solution. The lactic acid sensor 122 may transmit a first measured lactic acid concentration, which is the measured lactic acid concentration of the first standard solution, or an indicator thereof, to the controller 16. The first measured glucose concentration and the first measured lactic acid concentration may be stored in the storage unit 156. Using the information, the first standard solution measurement step may be brought to an end.

After the first standard solution measurement step, as illustrated FIG. 9, the biosensor 90 may be cleaned (step S18). For example, as illustrated in FIG. 7, the clamp control unit 160 may control the sampling clamp 146 and the first introduction clamp 148 to place them in an open state, and in the sampling flow path 98 a portion between the third branching section 110 and the fourth merging section 114 may be opened together with opening the first introduction flow path 100. The pump control unit 158 may also turn on the introduction pump 136.

Upon doing so, the cleaning solution may flow from the cleaning solution accommodation unit 92 to the waste liquid accommodation unit 22 via the first introduction flow path 100, the sampling flow path 98, and the third waste liquid flow path 76. During which, the biosensor 90 may be subjected to cleaning by the cleaning solution. When the cleaning of the biosensor 90 is completed, the pump control unit 158 may turn off the introduction pump 136. Further, the clamp control unit 160 may control the sampling clamp 146 and the first introduction clamp 148 to place them in a closed state, and in the sampling flow path 98, the portion between the third branching section 110 and the fourth merging section 114 may be closed together with closing the first introduction flow path 100.

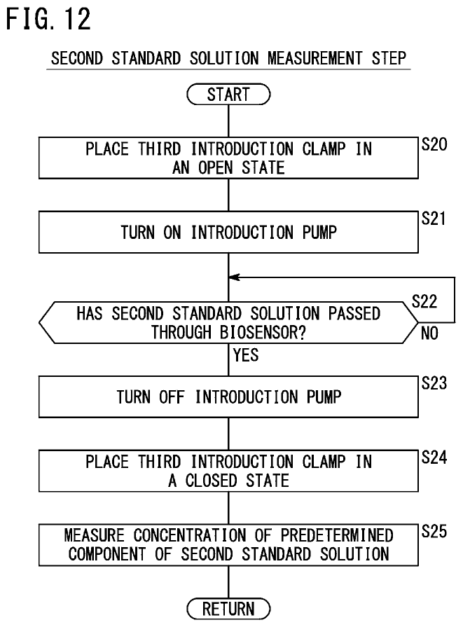

Thereafter, the second standard solution measurement step (step S19 of FIG. 9) may be carried out. In the second standard solution measurement step, as illustrated in FIG. 12, the clamp control unit 160 may control the third introduction clamp 152 to be placed in an open state, thereby causing the third introduction flow path 104 to open (step S20). Further, the pump control unit 158 may turn on the introduction pump 136 (step S21).

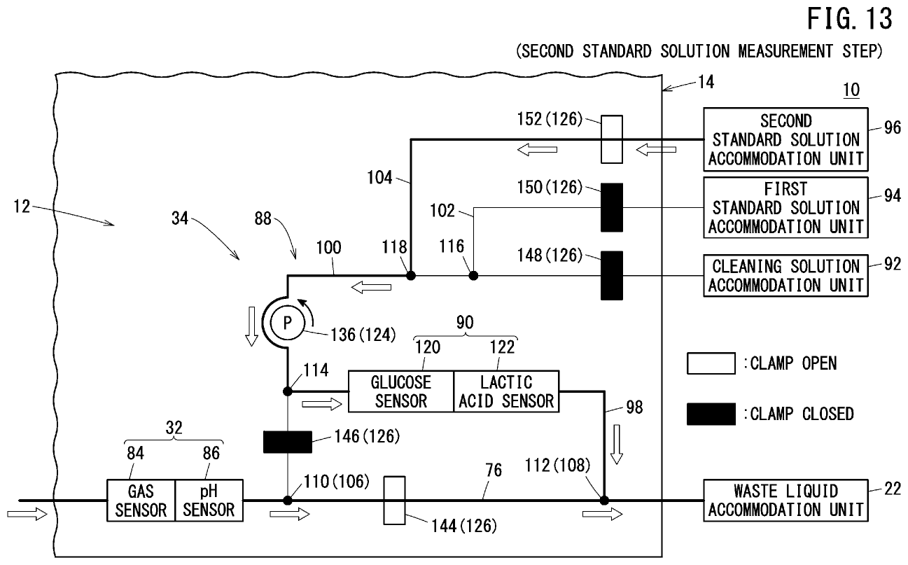

Upon doing so, as illustrated in FIG. 13, the second standard solution may flow from the second standard solution accommodation unit 96 into the waste liquid accommodation unit 22 via the third introduction flow path 104, the first introduction flow path 100, the sampling flow path 98, and the third waste liquid flow path 76. At this time, the determination unit 162 may determine whether or not the second standard solution has passed through the biosensor 90 (step S22 of FIG. 12).

The determination unit 162 may determine, for example, whether or not an elapsed time period since the introduction pump 136 has been turned on has reached a second standard solution introduction time period. The second standard solution introduction time period may be set based on the shape of the sampling unit 34, the size of the sampling unit 34, the flow rate of the second standard solution, and/or the like. The second standard solution introduction time period may be stored in the storage unit 156. The determination unit 162 may determine whether or not the second standard solution has passed through the biosensor 90 based on a detection signal of a second standard solution sensor that is configured to detect the second standard solution. The second standard solution sensor may be arranged, for example, in the sampling flow path 98 in a portion thereof between the biosensor 90 and the third merging section 112. In at least one example embodiment, however, the second standard solution sensor may be arranged in the third waste liquid flow path 76 in a portion thereof between the third merging section 112 and the waste liquid accommodation unit 22. Moreover, in at least one example embodiment, the cell culturing system 10 may include a single standard liquid sensor provided with the function of the first standard solution sensor and the function of the second standard solution sensor described above.

As illustrated in FIG. 12, when it is determined by the determination unit 162 that the second standard solution has not passed through the biosensor 90 (step S22: NO), the process of step S22 may be performed again. When it is determined by the determination unit 162 that the second standard solution has passed through the biosensor 90 (step S22: YES), the pump control unit 158 may turn off the introduction pump 136 (step S23). Further, the clamp control unit 160 may control the third introduction clamp 152 to be placed in a closed state, thereby causing the third introduction flow path 104 to close (step S24). In accordance therewith, flowing of the second standard solution of the biosensor 90 may be stopped.

The sensor control unit 164 may control the biosensor 90 to measure the concentration of a predetermined component of the second standard solution (step S25). For example, the sensor control unit 164 may control the glucose sensor 120 to measure the glucose concentration of the second standard solution. The glucose sensor 120 may transmit a second measured glucose concentration, which is the measured glucose concentration of the second standard solution, to the controller 16.

The sensor control unit 164 may control the lactic acid sensor 122, which is configured to measure the lactic acid concentration of the second standard solution. The lactic acid sensor 122 may transmit a second measured lactic acid concentration, which is the measured lactic acid concentration of the second standard solution, to the controller 16. The second measured glucose concentration and the second measured lactic acid concentration may be stored in the storage unit 156. In accordance therewith, the second standard solution measurement step may be brought to an end.

After the second standard solution measuring step, as illustrated in FIG. 9, the sensor correction unit 166 may correct the biosensor 90 (step S26). For example, the sensor correction unit 166 may calculate a first glucose deviation, which is the difference between the first measured glucose concentration and the first standard glucose concentration. Further, the sensor correction unit 166 may calculate a second glucose deviation, which is the difference between the second measured glucose concentration and the second standard glucose concentration. In addition, the sensor correction unit 166 may correct the measurement accuracy (measurement sensitivity) of the glucose sensor 120, for example, in a manner so that the first glucose deviation and the second glucose deviation are minimized.

The sensor correction unit 166 may calculate a first lactic acid deviation, which is the difference between the first measured lactic acid concentration and the first standard lactic acid concentration. Further, the sensor correction unit 166 may calculate a second lactic acid deviation, which is the difference between the second measured lactic acid concentration and the second standard lactic acid concentration. Thereafter, the sensor correction unit 166 may correct the measurement accuracy (measurement sensitivity) of the lactic acid sensor 122, for example, in a manner so that the first lactic acid deviation and the second lactic acid deviation are minimized. In accordance therewith, the biosensor calibration step may be brought to an end.

When the biosensor calibration step is carried out, the culture medium may continuously flow through the waste liquid flow path 62 toward the waste liquid accommodation unit 22. As such, during the biosensor calibration step, it may be possible to effectively prevent the first standard solution and the second standard solution from flowing back into the circulation flow paths 68.

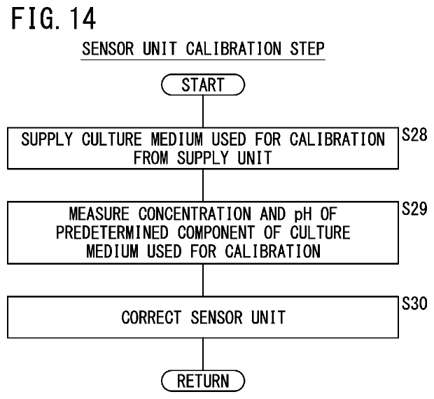

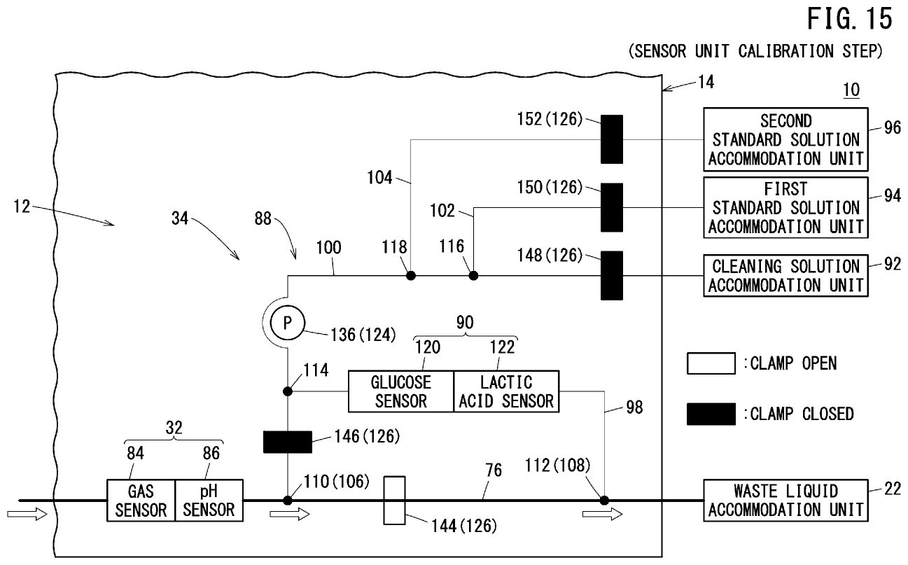

As illustrated in FIG. 4, the sensor unit calibration step may be carried out (step S27). In the sensor unit calibration step, as illustrated in FIG. 14, the controller 16 may control the supply unit 18 and may thereby cause the culture medium, which is used for calibration, to be supplied from the supply unit 18 to the culturing circuit 28 (step S28). Upon doing so, the culture medium used for calibration may flow through the sensor unit 32 (see FIG. 15). The oxygen concentration of the culture medium used for calibration may be set to a standard oxygen concentration. The carbon dioxide concentration of the culture medium used for calibration may be set to a standard carbon dioxide concentration. The pH of the culture medium used for calibration may be set to a standard pH.

As illustrated in FIG. 14, the sensor control unit 164 may control the sensor unit 32 to measure the concentration and the pH of a predetermined component of the culture medium used for calibration (step S29). For example, the sensor control unit 164 may control the oxygen sensor of the gas sensor 84 to measure the oxygen concentration of the culture medium used for calibration. The oxygen sensor may transmit the measured oxygen concentration, which is the measured oxygen concentration of the culture medium used for calibration, to the controller 16.

The sensor control unit 164 may control the carbon dioxide sensor of the gas sensor 84 to measure the carbon dioxide concentration of the culture medium used for calibration. The carbon dioxide sensor may transmit the measured carbon dioxide concentration, which is the measured carbon dioxide concentration of the culture medium used for calibration, to the controller 16. The sensor control unit 164 may control the pH sensor 86 to measure the pH of the culture medium used for calibration. The pH sensor 86 may transmit the measured pH, which is the measured pH of the culture medium used for calibration, to the controller 16.

The sensor correction unit 166 may correct the sensor unit 32 (step S30). For example, the sensor correction unit 166 may correct the measurement accuracy (measurement sensitivity) of the oxygen sensor in a manner so that the measured oxygen concentration becomes the standard oxygen concentration. The sensor correction unit 166 may correct the measurement accuracy (measurement sensitivity) of the carbon dioxide sensor in a manner so that the measured carbon dioxide concentration becomes the standard carbon dioxide concentration. The sensor correction unit 166 may correct the pH sensor 86 in a manner so that the measured pH becomes the standard pH. In accordance therewith, the sensor unit calibration step may be brought to an end.

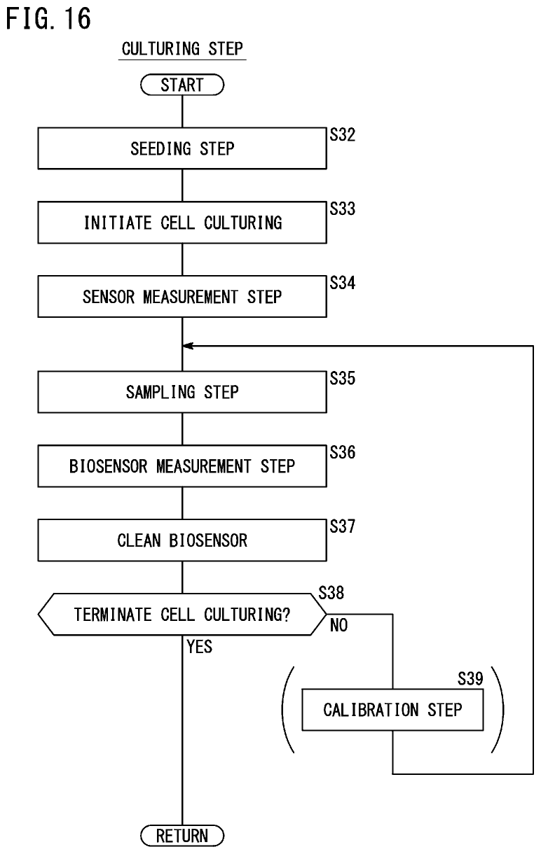

After the sensor unit calibration step, as illustrated in FIG. 4, the culturing step (step S31) may be carried out. In the culturing step, as illustrated in FIG. 16, a seeding step (step S32) may be performed. In the seeding step, the controller 16 may control the supply unit 18 to supply the cell solution from the supply unit 18 to the first supply flow path 52. Upon doing so, the cell solution may be introduced from the supply unit 18 into the first merging section 64 of the first circulation flow path 54 via the first supply flow path 52 (see FIG. 5). The cell solution having been introduced into the first merging section 64 may flow from the first inlet port 44 through the first region 40 and may be guided to the first outlet port 46. At this time, the cells within the cell solution may adhere to the inner surfaces of each of the hollow fiber membranes 36 of the bioreactor 26.

Next, cell culturing may be initiated (step S33). The controller 16 may control the supply unit 18 and may cause the culture medium to be supplied from the supply unit 18 to the first supply flow path 52. Upon doing so, the culture medium may be introduced from the supply unit 18 into the first merging section 64 of the first circulation flow path 54 via the first supply flow path 52 (see FIG. 5). The culture medium having been introduced into the first merging section 64 may circulate in the annular flow path including the first circulation flow path 54, the first inlet port 44, the first region 40, and the first outlet port 46.

The controller 16 may control the supply unit 18 to cause the culture medium to be supplied from the supply unit 18 to the second supply flow path 56. Upon doing so, the culture medium may be introduced from the supply unit 18 into the second merging section 66 of the second circulation flow path 58 via the second supply flow path 56. The culture medium having been introduced into the second merging section 66 may circulate in the annular flow path including the second circulation flow path 58, the second inlet port 48, the second region 42, and the second outlet port 50.

The gas exchange control unit 168 may control the gas exchange unit 30 to thereby carry out gas exchange on the culture medium flowing through the second circulation flow path 58. For example, a gas of a predetermined component may be passed through the culture medium prior to the culture medium flowing into the second inlet port 48. In accordance therewith, the gas concentration (e.g., the oxygen gas concentration and the carbon dioxide gas concentration) and the pH of the culture medium introduced into the second inlet port 48 of the bioreactor 26 can be adjusted to values suitable for cell culturing. In the bioreactor 26, the culture medium in the first region 40 and the culture medium in the second region 42 may be exchanged through the pores of each of the hollow fiber membranes 36. In accordance therewith, the gas concentration and the pH of the culture medium in the first region 40 may be adjusted.

Further, at an appropriate timing, the clamp control unit 160 may control the first waste liquid clamp 140, thereby causing the first waste liquid flow path 72 to open or close. When the first waste liquid flow path 72 is opened, a portion of the culture medium inside the first circulation flow path 54 may be guided to the third waste liquid flow path 76 via the first waste liquid flow path 72. Further, at an appropriate timing, the clamp control unit 160 may control the second waste liquid clamp 142, thereby causing the second waste liquid flow path 74 to open or close. When the second waste liquid flow path 74 is opened, a portion of the culture medium inside the second circulation flow path 58 may be guided to the third waste liquid flow path 76 via the second waste liquid flow path 74.

When cell culturing is initiated, the sensor measurement step may be started (step S34). In the sensor measurement step, the sensor control unit 164 may control the sensor unit 32 to measure the oxygen concentration, the carbon dioxide concentration, and the pH of the culture medium. The sensor unit 32 may transmit the measurement results to the controller 16. The sensor measurement step may be carried out until the culturing step is completed.

Next, the sampling step may be carried out (step S35). The sampling step may be carried out at an appropriate timing after the start of cell culturing. In the sampling step, for example, the clamp control unit 160 may control the first waste liquid clamp 140 to be placed in an open state, thereby causing the first waste liquid flow path 72 to open. Further, the clamp control unit 160 may control the second waste liquid clamp 142 to be placed in a closed state, thereby causing the second waste liquid flow path 74 to close. In this case, a portion of the culture medium flowing through the first circulation flow path 54 may be guided to the third waste liquid flow path 76 by the first circulation pump 130 via the first waste liquid flow path 72.

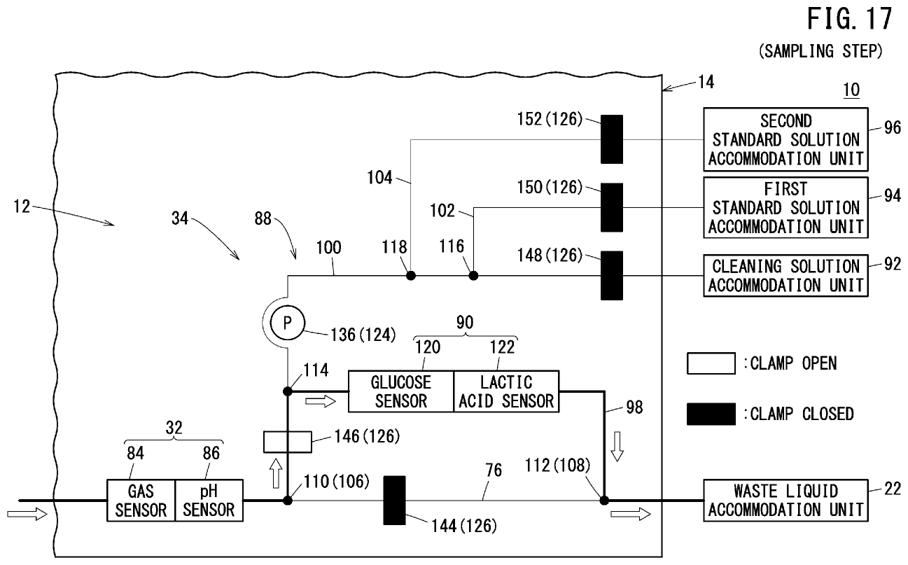

As illustrated in FIG. 17, the clamp control unit 160 may control the sampling clamp 146 to be placed in an open state, thereby causing a portion within the sampling flow path 98 between the third branching section 110 and the fourth merging section 114 to open. Furthermore, the clamp control unit 160 may control the third waste liquid clamp 144 to be placed in a closed state, thereby causing a portion within the third waste liquid flow path 76 between the third branching section 110 and the third merging section 112 to close. The pump control unit 158 may maintain the introduction pump 136 in an off state.

Upon doing so, the culture medium that was guided to the third waste liquid flow path 76 may flow from the third branching section 110 into the sampling flow path 98. The culture medium that has flowed into the sampling flow path 98 may flow into the waste liquid accommodation unit 22 via the biosensor 90, the third merging section 112, and the third waste liquid flow path 76.

In the sampling step, for example, the clamp control unit 160 may control the second waste liquid clamp 142 to be placed in an open state and may cause the second waste liquid flow path 74 to open. In this case, the clamp control unit 160 may control the first waste liquid clamp 140 to be placed in a closed state thereby causing the first waste liquid flow path 72 to close. Upon doing so, a portion of the culture medium flowing through the second circulation flow path 58 may be guided to the third waste liquid flow path 76 by the second circulation pump 134 via the second waste liquid flow path 74. For example, in the sampling step, either the culture medium flowing through the first circulation flow path 54 or the culture medium flowing through the second circulation flow path 58 can be collected in the sampling unit 34.

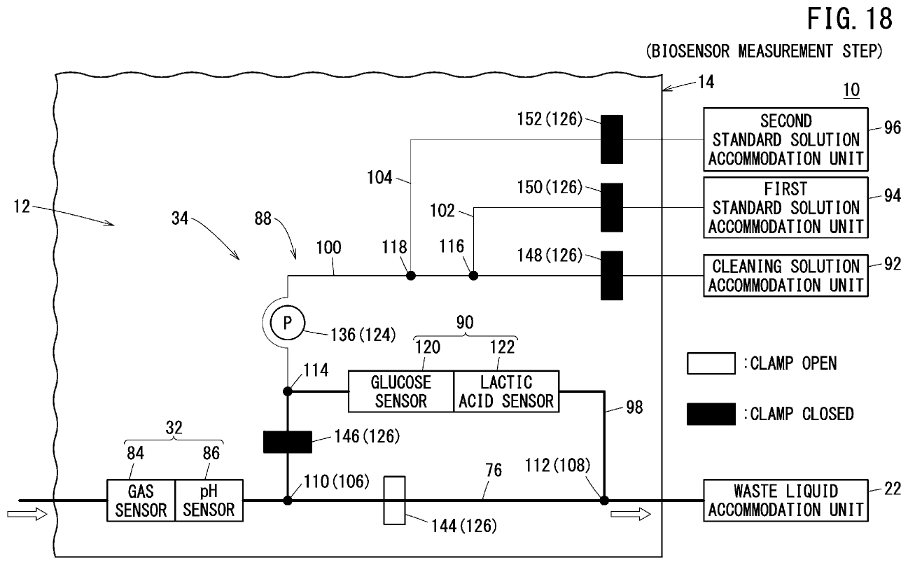

Thereafter, as illustrated in FIG. 16, a biosensor measurement step may be carried out (step S36). In the biosensor measurement step, as illustrated in FIG. 18, the clamp control unit 160 may control the sampling clamp 146 to be placed in a closed state thereby closing a portion in the sampling flow path 98 between the third branching section 110 and the fourth merging section 114. Further, the clamp control unit 160 may control the third waste liquid clamp 144 to be placed in an open state thereby causing a portion in the third waste liquid flow path 76 between the third branching section 110 and the third merging section 112 to open. In accordance therewith, flowing of the culture medium in the biosensor 90 can be stopped. Then, the sensor control unit 164 may control the biosensor 90 and may measure the glucose concentration and the lactic acid concentration of the culture medium. The biosensor 90 may transmit the measurement result to the controller 16.

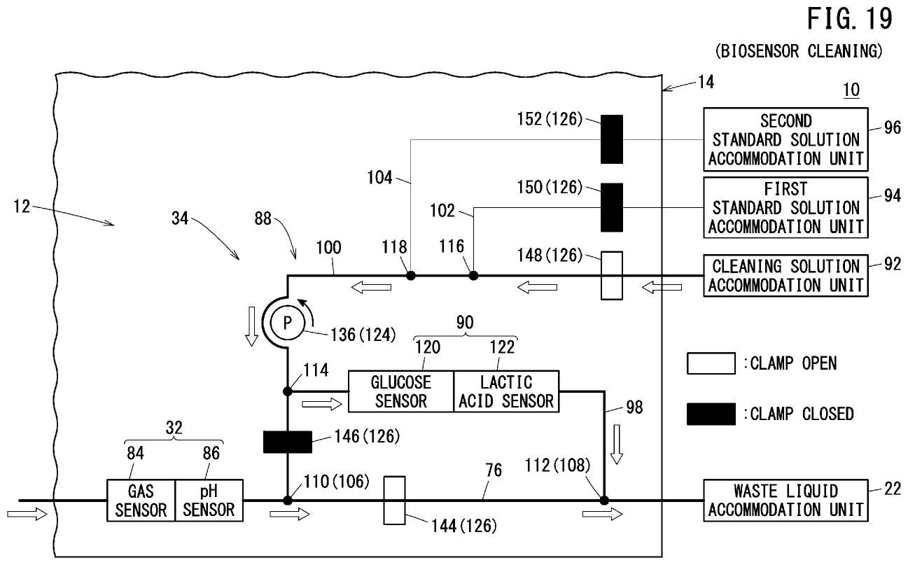

Thereafter, the biosensor 90 may be cleaned (step S37). For example, as illustrated in FIG. 19, the clamp control unit 160 may control the third waste liquid clamp 144 to be placed in an open state thereby causing a portion in the third waste liquid flow path 76 between the third branching section 110 and the third merging section 112 to open. Further, the clamp control unit 160 may control the first introduction clamp 148 to be placed in an open state thereby causing the first introduction flow path 100 to open. Further, the pump control unit 158 may turn on the introduction pump 136.

Upon doing so, the cleaning solution may flow from the cleaning solution accommodation unit 92 to the waste liquid accommodation unit 22 via the first introduction flow path 100, the sampling flow path 98, and the third waste liquid flow path 76. In accordance therewith, the biosensor 90 may be subjected to cleaning by the cleaning solution.

As illustrated in FIG. 16, the controller 16 may determine whether or not to terminate the culturing of the cells based on the measurement result that was measured in the biosensor measurement step (step S38). When it is determined by the controller 16 that the culturing of the cells is not completed (step S38: NO), the calibration step (step S39) may be carried out. In the calibration step, the same processes as performed in the biosensor calibration step (step S10) described above may be carried out. After completion of the calibration step, the processes of step S35 may be performed. In at least one example embodiment, the calibration step may be omitted. The calibration step may be performed only when necessary.

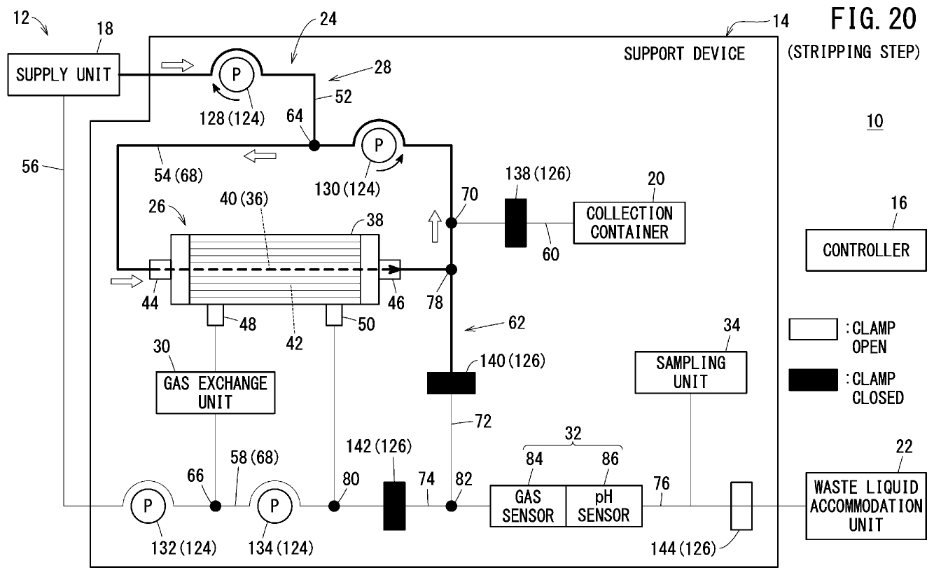

When it is determined by the controller 16 that the culturing of the cells is completed (step S38: YES), the stripping step in FIG. 4 may be carried out (step S40). In the stripping step, as illustrated in FIG. 20, the clamp control unit 160 may control the first waste liquid clamp 140 to be placed in a closed state thereby causing the first waste liquid flow path 72 to close. Further, the controller 16 may control the supply unit 18 to cause the stripping solution to be supplied from the supply unit 18 to the first supply flow path 52. At this time, the pump control unit 158 may turn off the second supply pump 132 and the second circulation pump 134.

Upon doing so, the stripping solution may be guided from the supply unit 18 to the bioreactor 26 via the first supply flow path 52 and the first circulation flow path 54. In the bioreactor 26, the stripping solution may strip the cultured cells from the inner surfaces of each of the hollow fiber membranes 36.

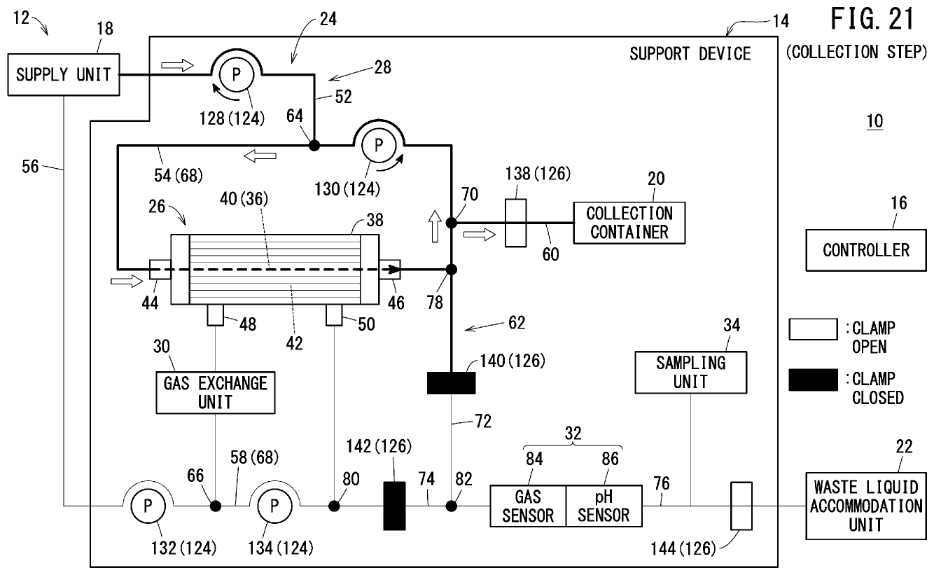

Thereafter, as illustrated in FIG. 4, the collection step may be carried out (step S41). In the collection step, as illustrated in FIG. 21, the clamp control unit 160 may control the collection clamp 138 thereby causing the collection flow path 60 to open. Upon doing so, a solution containing the cells inside the first circulation flow path 54 may be guided via the collection flow path 60 to the collection container 20. In accordance therewith, the flow of operations of the cell culturing method may come to an end.

The cell culturing method is not limited to the example described above. For example, the biosensor calibration step (step S10) and/or the second standard solution measurement step (step S19) illustrated in FIG. 9 may be omitted. In such instances case, in step S26, the sensor correction unit 166 may correct the biosensor 90 on the basis of the measurement result acquired, for example, during the first standard solution measurement step. Further, in FIG. 4, in the cell culturing method, the biosensor calibration step (step S10) and/or the sensor unit calibration step (step S27) may be omitted. Furthermore, in the cell culturing method, a third standard solution measurement step may be added. In the cell culturing method, the number of the standard solution measurement steps can be set in an appropriate manner. The cell culturing device 12 cultures cells by allowing a culture medium to flow from the circulation flow paths 68 to and through the bioreactor 26 connected therewith. The cell culturing device 12 may include the waste liquid flow path 62 and the waste liquid accommodation unit 22. The waste liquid flow path 62 may be a flow path that connects to the circulation flow paths 68 for discarding the liquid that flows through the circulation flow paths 68. The waste liquid accommodation unit 22 may accommodate the liquid guided from the waste liquid flow path 62. The sampling unit 34 may collect the culture medium that is guided from the circulation flow paths 68 to the waste liquid flow path 62 and may be connected to the waste liquid flow path 62.

In at least one example embodiment, the sampling unit 34 may be connected to the waste liquid flow path 62. The liquid may always flow in a single direction, for example, from the bioreactor 26 toward the waste liquid accommodation unit 22. As such, it is not necessary to attach an aseptic filter to the sampling unit 34 in order to maintain the aseptic state of the interior of the bioreactor 26, simplifying the configuration of the cell culturing device 12. Further, since a negative pressure is not generated in the flow paths as a result of clogs in an aseptic filter, it is possible to prevent the liquid inside the sampling unit 34 from flowing back into the circulation flow paths 68 through the waste liquid flow path 62.

The sampling unit 34 may include the sampling flow path 98 and the biosensor 90. The sampling flow path 98 may be connected to the waste liquid flow path 62. The biosensor 90 may be installed in the sampling flow path 98.

In accordance with such a configuration, the culture medium flowing through the waste liquid flow path 62 can flow into the sampling flow path 98 and be guided to the biosensor 90.

The sampling flow path 98 may have the first end 106 and the second end 108. The first end 106 may be connected to the waste liquid flow path 62. The second end 108 may be connected to a portion in the waste liquid flow path 62 between the first end 106 and the waste liquid accommodation unit 22. The biosensor 90 may be installed in an intermediate portion of the sampling flow path 98 so as to be in contact with the culture medium.

In accordance with such a configuration, the culture medium having passed through the biosensor 90 can be guided into the waste liquid accommodation unit 22. As a result, there may be no need to install in the sampling flow path 98 a culture medium accommodation unit used for disposal in order to accommodate the culture medium that has flowed through the biosensor 90, allowing the configuration of the cell culturing device 12 to be simplified.

The sampling unit 34 may include the cleaning solution accommodation unit 92 and the first introduction flow path 100. The cleaning solution may be accommodated in the cleaning solution accommodation unit 92. The first introduction flow path 100 may mutually connect the cleaning solution accommodation unit 92 and a portion in the sampling flow path 98 between the first end 106 and the biosensor 90.

In accordance with such a configuration, the cleaning solution can be guided from the cleaning solution accommodation unit 92 to the biosensor 90 via the first introduction flow path 100 and the sampling flow path 98. As a result, any of the culture medium that remains adhered to the biosensor 90 can be cleaned off by the cleaning solution, allowing the useful lifetime of the biosensor 90 to be maintained over a prolonged period.

The gas sensor 84, which measures a concentration of a gas component of the culture medium, may be installed at a portion in the waste liquid flow path 62 between the circulation flow paths 68 and the sampling unit 34.

When the concentration of the gas component of the culture medium is measured by the gas sensor 84, if the cleaning solution becomes mixed in the interior of the gas sensor 84, the measured value of the gas sensor 84 may change significantly. That is, there is a possibility that the gas sensor 84 may be incapable of accurately measuring the concentration of the gas component of the culture medium. However, when the biosensor 90 is cleaned by the cleaning solution, the cleaning solution does not flow through the gas sensor 84. Specifically, in accordance with the present disclosure, the cleaning solution does not become mixed inside the gas sensor 84. Accordingly, the gas sensor 84 is capable of accurately measuring the concentration of the gas component of the culture medium.

The bioreactor 26 may include the plurality of hollow fiber membranes 36. The housing 38 may be configured to accommodate the plurality of hollow fiber membranes 36. The circulation flow paths 68 may include the first circulation flow path 54 and the second circulation flow path 58. The first circulation flow path 54 may communicate with the inner holes (the first region 40) of the plurality of hollow fiber membranes 36. The second circulation flow path 58 may communicate with the second region 42 between the plurality of hollow fiber membranes 36 and the housing 38. Each of the plurality of hollow fiber membranes 36 may be configured in a manner so that the culture medium is capable of being exchanged between the first region 40 and the second region 42. The waste liquid flow path 62 may include the first waste liquid flow path 72, the second waste liquid flow path 74, and the third waste liquid flow path 76. The first waste liquid flow path 72 may be connected to the first circulation flow path 54. The second waste liquid flow path 74 may be connected to the second circulation flow path 58. The third waste liquid flow path 76 may be connected to the waste liquid accommodation unit 22. The third waste liquid flow path 76 may include the intermediate merging section 82 where the first waste liquid flow path 72 and the second waste liquid flow path 74 merge.

The sampling unit 34 may be connected to the third waste liquid flow path 76.

In at least one example embodiment, either the culture medium which has flowed through the first region 40, or the culture medium which has flowed through the second region 42, can be selected and then collected by the sampling unit 34.

The sampling flow path 98 may be aseptically joined to the third waste liquid flow path 76.

In accordance with such a configuration, it may be possible to prevent bacteria from being mixed in the interior of the cell culturing device 12 from a joined portion between the sampling flow path 98 and the third waste liquid flow path 76.

The cell culturing system 10 may include the cell culturing device 12 and the support device 14. The cell culturing device 12 may be detachably installed on the support device 14. The wall portion constituting the flow paths of the cell culturing device 12 may be flexible. The support device 14 may include the plurality of clamps 126 that press on the outer surface of the wall portion and that are configured to cause the flow paths of the cell culturing device 12 to close.

In accordance with such a configuration, since it is unnecessary to incorporate the clamps 126 into the cell culturing device 12, the configuration of the cell culturing device 12 can be simplified. For example, the manufacturing cost of the disposable cell culturing device 12 can be reduced.

In the cell culturing system 10, the plurality of clamps 126 may include the third waste liquid clamp 144 and the sampling clamp 146. In the set state, the third waste liquid clamp 144 may be positioned in the third waste liquid flow path 76 at a portion between the first end 106 and the second end 108.

In the set state, the sampling clamp 146 may be disposed in the sampling flow path 98 at a portion between the first end 106 and the biosensor 90.

In accordance with such a configuration, by placing the third waste liquid clamp 144 in a closed state together with placing the sampling flow path 98 in an open state, the culture medium in the third waste liquid flow path 76 can be allowed to flow through the biosensor 90. Further, by placing the third waste liquid clamp 144 in an open state together with placing the sampling clamp 146 in a closed state, the culture medium in the third waste liquid flow path 76 can be guided to the waste liquid accommodation unit 22 without flowing through the biosensor 90.

In at least one example embodiment, the sensor unit 32 may be installed in the sampling flow path 98 instead of the third waste liquid flow path 76. The sampling unit 34 may be connected to either the first waste liquid flow path 72 or the second waste liquid flow path 74 instead of the third waste liquid flow path 76. The same advantageous effects as those of the above-described sampling unit 34 are exhibited. For example, when the sampling unit 34 is connected to the first waste liquid flow path 72, the sampling unit 34 may be capable of collecting the culture medium that has flowed through the first region 40. When the sampling unit 34 is connected to the second waste liquid flow path 74, the sampling unit 34 may be capable of collecting the culture medium that has flowed through the second region 42.

The cell culturing device 12 may include the calibration sensor for measuring the glucose concentration and the lactic acid concentration in the culture medium. During which, in the biosensor calibration step, the sensor correction unit 166 may correct the measurement accuracy (measurement sensitivity) of the biosensor 90 based on the measured value of the calibration sensor. Therefore, in the cell culturing device 12, the first standard solution accommodation unit 94, the second standard solution accommodation unit 96, the second introduction flow path 102, and the third introduction flow path 104 can be omitted.

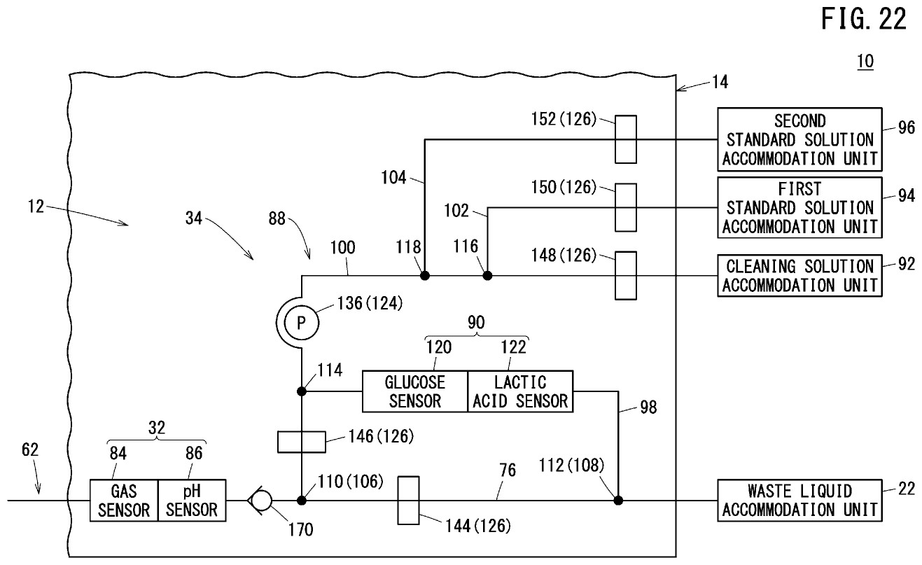

As illustrated in FIG. 22, in the cell culturing device 12, a check valve 170 may be installed in a portion in the third waste liquid flow path 76 between the third branching section 110 and the sensor unit 32. The check valve 170 may be installed which allows flowing of the liquid in a direction toward the waste liquid accommodation unit 22 and which prevents flowing of the liquid in a direction toward the circulation flow paths 68. The check valve 170 may be installed in any position in the waste liquid flow path 62 as long as the position is on a more upstream side than the portion (the third branching section 110) to which the sampling unit 34 is connected.

In at least one example embodiment, as illustrated in FIG. 22, on the waste liquid flow path 62, upstream of a portion (the third branching section 110) to which the sampling unit 34 is connected, the check valve 170 may be installed that allows flowing of the liquid in a direction toward the waste liquid accommodation unit 22 and may be configured to prevent flowing of the liquid in a direction toward the circulation flow paths 68.

In accordance with such a configuration, any risk of bacteria from the sampling unit 34 entering into and contaminating the bioreactor 26 can be reduced by the check valve 170.

The present disclosure is not limited to the embodiment described above and various alternative configurations could be adopted therein without deviating from the essence and gist of the present disclosure.

In various aspects, the present disclosure provides a cell culturing device 12 that cultures cells, for example, by allowing culture medium to flow inside the bioreactor 26. The culture medium may flow to the bioreactor 26 from a circulation flow path 68. The cell culturing device 12 includes, for example, a waste liquid flow path 62 connected to the circulation flow path 68, the waste liquid flow path 62 allowing liquid flowing through the circulation flow path 68 to be discarded. The cell culturing device 12 may also include, for example, a waste liquid accommodation unit 22 in which the liquid guided from the waste liquid flow path 62 may be accommodated. The cell culturing device 12 may also include a sampling unit 34 that is configured to collect the culture medium guided, for example, from the circulation flow path 68 and to the waste liquid flow path 62. The sampling unit 34 may be connected to the waste liquid flow path 62.

In at least one example embodiment, the sampling unit 34 may include a sampling flow path 98 that is connected to the waste liquid flow path 62. The sampling unit 34 may also include a biosensor 90 installed, for example, in the sampling flow path 98.

In at least one example embodiment, a first end 106 of the sampling flow path 98 may be connected to the waste liquid flow path 62 and a second end 108 connected to a portion of the waste liquid flow path 62 between the first end 106 and the waste liquid accommodation unit 22. The biosensor 90 may be installed in an intermediate portion of the sampling flow path 98 so as to be in contact with the culture medium.

In at least one example embodiment, the sampling unit 34 may include a cleaning solution accommodation unit 92 in which the cleaning solution is accommodated. The sampling unit 34 may also include an introduction flow path 100 that connects the cleaning solution accommodation unit 92 and the portion in the sampling flow path 98 between the first end 106 and the biosensor 90.

In at least one example embodiment, the waste liquid flow path 62 may include a sensor unit 32 that measures at least one of the concentration and the pH of a gas component of the culture medium. The sensor unit 32 may be disposed between the circulation flow path 68 and the sampling unit 34.

In at least one example embodiment, the bioreactor 26 may include a plurality of hollow fiber membranes 36 and a housing 38 that is configured to accommodate the plurality of hollow fiber membranes 36. The circulation flow path 68 may include a first circulation flow path 54 that is in communication with a first region 40 defined by inner holes of the plurality of hollow fiber membranes 36. The circulation flow path 68 may also include a second circulation flow path 58 that is in communication with a second region 42 defined by the space between the plurality of hollow fiber membranes 36 and the housing 38. Each of the plurality of hollow fiber membranes 36 may be configured in a manner so that the culture medium is capable of being exchanged between the first region 40 and the second region 42. The waste liquid flow path 62 may include a first waste liquid flow path 72 that may be connected to the first circulation flow path 54, a second waste liquid flow path 74 that may be connected to the second circulation flow path 58, and a third waste liquid flow path 76 that may be connected to the waste liquid accommodation unit 22. The third waste liquid flow path 76 may include an intermediate merging section 82 where the first waste liquid flow path 72 and the second waste liquid flow path 74 merge.

In at least one example embodiment, the sampling unit 34 may be connected to the third waste liquid flow path 76.

In at least one example embodiment, the waste liquid flow path 62 may include a check valve 170. The check valve 170 may be disposed to a portion of the waste liquid flow path 62 upstream to a portion of the waste liquid flow path 62 to which the sampling unit 34 is connected. The check valve 170 may be installed to allow flowing of the liquid in a direction toward the waste liquid accommodation unit 22. The check valve 170 may help to prevent flowing of the liquid in a direction toward the circulation flow path.

In at least one example embodiment, the sampling unit 24 and the waste liquid flow path 62 may be aseptically joined.

In various aspects, the present disclosure provides a cell culturing system 10. The cell culturing system 10 may include the aforementioned cell culturing device 12 and the support device 14 to which the cell culturing device 12 may be detachably installed, where the wall portions of the flow paths of the cell culturing device 12 are flexible, and the support device 14 includes a plurality of clamps 126 that are configured to press the outer surface of the wall portion and cause the flow paths of the cell culturing device 12 to close.

In at least one example embodiment, the sampling unit 34 may include the sampling flow path 98 connected to the waste liquid flow path 62. The biosensor 90 may be attached to the sampling flow path 98. The sampling flow path 98 may include the first end 106 connected to the waste liquid flow path 62, and the second end 108 connected to a portion in the waste liquid flow path 62 between the first end 106 and the waste liquid accommodation unit 22. The biosensor 90 may be attached to the intermediate portion of the sampling flow path 98 so as to be in contact with the culture medium. The plurality of clamps 126 may include, in the set state in which the cell culturing device 12 is attached to the support device 14, the waste liquid clamp 144 positioned in the waste liquid flow path 62 at a portion between the first end and the second end, and in the set state, the sampling clamp 146 positioned in the sampling flow path 98 at a portion between the first end and the biosensor.

1. A cell culturing device comprising:
a circulation flow path in fluid communication with a bioreactor and configured for flow of a liquid therein;
a waste liquid flow path in fluid communication with the circulation flow path and configured to receive a discard portion of the liquid from the circulation flow path;
a waste liquid accommodation unit in fluid communication with the waste liquid flow path and configured to receive and hold at least a portion of the discard portion of the liquid from the waste liquid flow path; and
a sampling unit in fluid communication with the waste liquid flow path between the circulation flow path and the waste liquid accommodation unit.

2. The cell culturing device of claim 1, wherein the sampling unit comprises:
a sampling flow path in fluid communication with the waste liquid flow path; and
a biosensor disposed along the sampling flow path.

3. The cell culturing device of claim 2, wherein the biosensor includes a glucose sensor, a lactic acid sensor, a glutamic acid sensor, or any combination thereof.

4. The cell culturing device of claim 2, wherein the sampling flow path includes:
a first end in fluid communication with the waste liquid flow path; and
a second end in fluid communication with the waste liquid flow path between the first end and the waste liquid accommodation unit,
wherein the biosensor is disposed between the first end and the second end of the sampling flow path.

5. The cell culturing device of claim 4, wherein the sampling unit includes:
a cleaning solution accommodation unit configured to hold a cleaning solution; and
an introduction flow path in fluid communication with the cleaning solution accommodation unit and also in fluid communication with the sampling flow path between the first end and the biosensor.

6. The cell culturing device of claim 1, wherein the cell culturing device further includes:
a sensor unit in fluid communication with the waste liquid flow path between the circulation flow path and the sampling unit.

7. The cell culturing device of claim 6, wherein the sensor unit is configured to measure a concentration of a gas component of the liquid, to measure a pH of the gas component of the liquid, or to measure the concentration and the pH of the gas component.

8. The cell culturing device of claim 1, wherein the waste liquid flow path includes:
a check valve configured to allow the liquid to move in a first direction toward the waste liquid accommodation unit and prevents movement of the liquid in a second direction toward the circulation flow path.

9. The cell culturing device of claim 8, wherein the check valve is disposed between the sensor unit and the sampling unit.

10. The cell culturing device of claim 1, wherein
the bioreactor includes:
a plurality of hollow fiber membranes; and
a housing configured to receive the plurality of hollow fiber membranes; and
the circulation flow path includes:
a first circulation flow path in communication with a first region of the plurality of hollow fiber membranes; and
a second circulation flow path in communication with a second region of the plurality of hollow fiber membranes, the plurality of hollow fiber membranes being configured to allow the liquid to travel between the first region and the second region.

11. The cell culturing device of claim 10, wherein the first region is defined by interior surfaces of the plurality of hollow fiber membranes, and the second region is defined by a space between the plurality of hollow fiber membranes and an interior-surface of the housing.

12. The cell culturing device of claim 10, wherein the waste liquid flow path includes:
a first waste liquid flow path in fluid communication with the first circulation flow path;
a second waste liquid flow path in fluid communication with the second circulation flow path; and
a third waste liquid flow path in fluid communication with the first and second waste liquid flow paths and also the waste liquid accommodation unit.

13. The cell culturing device of claim 6, wherein the sampling unit is in fluid communication with the third waste liquid flow path.

14. The cell culturing device of claim 1, wherein the sampling unit and the waste liquid flow path are aseptically joined.

15. A cell culturing system comprising:
a cell culturing device including:
a circulation flow path in fluid communication with a bioreactor and configured for flow of a liquid therein;
a waste liquid flow path in fluid communication with the circulation flow path and configured to receive a discard portion of the liquid from the circulation flow path;
a waste liquid accommodation unit in fluid communication with the waste liquid flow path and configured to receive and hold at least a portion of the discard portion of the liquid from the waste liquid flow path; and
a sampling unit in fluid communication with the waste liquid flow path between the circulation flow path and the waste liquid accommodation unit; and
a support device configured to receive the cell culturing device, the support device including a plurality of clamps configured to apply a pressure to one or more portions of the circulation flow path or the waste liquid flow path.

16. The cell culturing system of claim 15, wherein the sampling unit includes:
a sampling flow path in fluid communication with the waste liquid flow path, the sampling flow path including a first end in fluid communication with the waste liquid flow path and a second end in fluid communication with the waste liquid flow path between the first end and the waste liquid accommodation unit; and
a biosensor disposed along the sampling flow path between the first end and the second end.

17. The cell culturing system of claim 16, wherein the plurality of clamps includes:
a waste liquid clamp disposed to selectively apply a pressure to the waste liquid flow path between the first end and the second end.

18. The cell culturing system of claim 16, wherein the plurality of clamps includes:
a sampling clamp disposed to selectively apply a pressure to the sampling flow path between the first end and the biosensor.

19. The cell culturing system of claim 15, wherein the cell culturing device further includes:
a sensor unit in fluid communication with the waste liquid flow path between the circulation flow path and the sampling unit.

20. The cell culturing device of claim 19, wherein the waste liquid flow path includes:
a check valve configured to allow the liquid to move in a first direction toward the waste liquid accommodation unit and prevents movement of the liquid in a second direction toward the circulation flow path, the check valve being disposed between the sensor unit and the sampling unit.
